(19) United States
(12) Patent Application Publication
(10) Pub. No.: US 2009/0124579 A1
(43) Pub. Date: May 14, 2009

(54) Systems and Methods For Determination of Compounds for Stimulation or Inhibition of Neocalcification

(75) Inventors: Brian Genge, Columbia, SC (US); Licia Wu, Cayce, SC (US); Roy Wuthier, Columbia, SC (US)

Correspondence Address: DORITY & MANNING, P.A., POST OFFICE BOX 1449, GREENVILLE, SC, 29602-1449, US

(73) Assignee: UNIVERSITY OF SOUTH CAROLINA, Columbia, SC (US)

(21) Appl. No.: 12/266,837

(22) Filed: Nov. 7, 2008

Related U.S. Application Data
(60) Provisional application No. 61/002,552, filed on Nov. 9, 2007.

(51) Int. Cl. A61K 31/685; A61K 31/28; A61K 31/285

(52) U.S. Cl. 514/77; 514/114; 514/492; 514/504

(57) ABSTRACT

Disclosed are systems and methods as may be used for development of compounds that can be utilized for prevention of pathological calcification. Disclosed are functional models can represent in great detail the first steps in protein-membrane interaction leading to calcification. Disclosed compounds include those that interfere with pathologic calcification processes and thus may be used as part of a formulation or composition for preventing pathological calcifications. Modeling methods and systems can include in vitro and/or in silico dynamic molecular modeling of biological materials including representative mineralization models in conjunction with substances that can affect normal or pathogenic calcification processes. Disclosed models can examine interactions of biological components involved in calcification including calcium, inorganic phosphate, phosphatidylserine, and annexin-5 protein.

### CROSS REFERENCE TO RELATED APPLICATION

The present application claims filing benefit of U.S. Provisional Patent Application Ser. No. 61/002,552 having a filing date of Nov. 9, 2007, which is incorporated herein in its entirety.

### GOVERNMENT SUPPORT CLAUSE

This invention was developed with funding from the following agencies:

Office of Naval Research Grant N00014-97-1-0806 and National Institute of Arthritis and Musculoskeletal and Skin Diseases Grants AR18983 and AR42359. Therefore, the government retains certain rights in this invention.

### BACKGROUND

In normal growth plate development, matrix vesicles (MV) form when hypertrophic chondrocytes combine calcium ion (Ca²⁺), inorganic phosphate (Pi) and other mineral ions with key proteins and lipids, enclose them within a lipid bilayer, and shed them from the cell surface into the extracellular matrix. This cell-mediated process produces unique nanomaterials, nucleational complexes capable of inducing de novo formation of a biomineral that is subsequently propagated to calcify the developing appendicular skeleton. Studies have identified amorphous calcium phosphate (ACP), phosphatidylserine (PS), and the protein annexin 5 as key constituents of these nucleational complexes.

Biological calcification in vertebrates is a cell-mediated physiological process by which tissues become calcified by the precipitation of calcium phosphate salts. It occurs normally during development of hard tissues, e.g. during endochondral calcification in long-bone formation, or recapitulation of this in the healing fracture callus. In contrast, pathological calcification occurs in diseases such as osteoarthritis, chondrocalcinosis, and atherosclerosis where it often occurs in association with atherosclerotic plaques. Pathological calcification is correlated with arterial wall rigidity, increased pulse pressure and cardiac work that contribute to the high mortality rates in heart disease. While matrix vesicle (MV) mediated processes are clearly involved, a variety of other factors including bone morphogenetic proteins, metalloproteases and cellular processes also contribute to pathological calcification.

Endochondral ossification is the process by which a cartilage anlagen grows, calcifies, and is replaced by bone. In the growth plate, bone elongation results from proliferation and hypertrophy of growth plate chondrocytes, accompanied by deposition of an extracellular matrix composed largely of proteoglycans and type II collagen. During the maturation stage, chondrocytes cease to divide and express additional genes. The cells load Ca²⁺ into their mitochondria; this blocks ATP synthesis and causes ATP-depletion. In the hypertrophic stage, the cells become enlarged (i.e. auxetic) and have elevated levels of Pi (20-25 mM); cytoplasmic Ca²⁺ also increases (400-500 nM). Near the end of this phase, a major influx of Ca²⁺ causes the Pi-loaded cells to undergo a type of apoptosis in which MV form. Ca²⁺ loading also leads to formation of PS.Ca.Pi complexes in the plasma membrane, mediated in part by the annexins. MVs bud from the plasma membrane at sites of interaction with the extracellular matrix, conveying to the extracellular matrix Ca²⁺, Pi, PS.Ca.Pi complex, and the annexins which together nucleate mineral formation. Following calcification of the cartilage matrix, ossification ensues via recruitment of osteoblasts, osteoclasts, and hematopoietic cells from the penetrating vessels. Osteoblasts deposit trabecular bone onto the mineralized cartilage; osteoclasts remodel this composite; and an influx of hematopoietic cells expands the bone marrow. This coordinated sequence of chondrocyte proliferation, maturation, hypertrophy, calcification, and apoptosis results in precise control of longitudinal growth and mineralization of bone.

MVs play a central role in de novo mineral formation in most vertebrate calcifying tissues. They induce mineral formation not only during longitudinal bone growth, but also in fracture healing and mantle dentine formation. Ectopic calcification also can be initiated by MV-like structures, contributing to the pathogenesis of arteriosclerosis and osteoarthritis. For example, MVs present in articular cartilage deposit hydroxyapatite crystals that promote cartilage degradation in osteoarthritis. In degenerative arthritis, an increase in chondrocyte apoptosis and expression of annexin 5 occur at sites of matrix calcification. In chondrocalcinosis a second type of MVs promotes formation of calcium pyrophosphate crystals. MVs appear to induce vascular calcification, leading to intramembranous-like bone formation in the intimal wall of the artery.

What are needed in the art are systems and methods for examining the molecular processes involved in calcification so as to develop compounds for preventing pathological calcification according to less expensive, more efficient methods. What are also needed are compounds to encourage calcification, for instance in bone healing applications that are specifically designed for the desired use and offer less systemic danger to a patient.

### SUMMARY

According to one embodiment, disclosed are methods and systems for identifying a test compound that effects formation of a complex including calcium ion, inorganic phosphate, and phosphatidylserine. For example, a method can include integrating physical parameters of components involved in neocalcification into an in silico system. Components can include, for example, calcium ion, inorganic phosphate, phosphatidylserine, annexin-5 protein, and a test compound. Physical parameters included in the in silico models can generally include the partial charges of atoms of the components the bond length between atoms of the components, the bond angles, and the dihedrals of the components. During a simulation, the components can be within an interactive distance of one another in order to determine the effect of the test compound on calcification. An interactive distance can be between about 5 and about 16 Angstroms (Å), or between about 10 and about 12 Å. For instance, it can be determined whether or not the test compound blocks or encourages formation of a complex comprising calcium ion, inorganic phosphate, and phosphatidylserine. In another embodiment, it can be determined whether or not a test compound effects the formation of an annexin-5 trimer through, for instance, the formation of the proper conformation of the protein, the formation of the trimer, and/or the interaction of the trimer with a phospholipid bilayer.

A modeling system can include additional components found in an in vivo calcification, including water, magnesium, carbonate, potassium, and so forth.

A method can combine an in silico process with an in vitro process. For instance, following the examination of a test compound in an in silico model, the components can be tested in an in vitro system.

According to another embodiment, disclosed are compounds and formulations including the compounds that can inhibit calcification. For example, in one embodiment, a formulation can include a low molecular analog of a phospholipid, for example, phosphatidylserine. Examples of low molecular analogs encompassed herein can include o-phospho-L-serine, 2-amino-4-phosphonobutyric acid, and compounds having the structure:

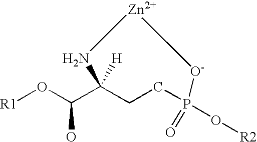

wherein R1 and R2 are independently H, CH₃, or a substituted or unsubstituted acyl chain up to about 20 carbons in length.

Other compounds disclosed herein include analogues of other lipids, phosphate analogues, small molecules, molecules incorporating divalent cations, and so forth.

### BRIEF DESCRIPTION OF THE FIGURES

A full and enabling disclosure of the present invention, including the best mode thereof to one skilled in the art, is set forth more particularly in the remainder of the specification, which includes reference to the accompanying figures, in which:

FIG. 1 is a schematic illustration of in vivo neocalcification in a matrix vesicle.

FIG. 2 is a transmission electron micrograph (TEM) of matrix vesicles isolated from growth plate cartilage;

FIG. 3 is a schematic block diagram of exemplary hardware components for implementing steps of the present signal processing methodology;

FIG. 4 illustrates the three-dimensional structure of avian annexin 5 (Anx-A5);

FIG. 5 illustrates the alignment of 3D structures of avian and human Anx-5;

FIGS. 6A and 6B illustrate the multiconductance Ca²⁺ channels formed upon addition of MV and Anx-A5 to planar PS.PE (phosphatidylethanolamine) (FIG. 6A) and the complete quenching of these channels by Zn²⁺ (FIG. 6B);

FIGS. 7A and 7B illustrate the trimer form of Anx-A5.Ca²⁺ (FIG. 7A) and the rearranged form of a trimer formed of Anx-A5.Zn²⁺ (FIG. 7B);

FIG. 8 schematically illustrates the Anx-5 trimer by domain;

FIG. 9A compares the mineral formation by avian MV and synthetic PS.Ca.Pi complexes in the presence and absence of human annexin (Anx-H5) when incubated in synthetic cartilage lymph (SCL);

FIG. 9B illustrates the x-ray confirmation of mineral formation in the systems of FIG. 9A;

FIG. 10A illustrates ball and stick and licorice molecular models of PS and its rearrangement upon Ca²⁺ and Pi binding to form the PS.Ca.Pi complex;

FIG. 10B illustrates the polar head group of PS with Ca²⁺ and Pi bound to key functional groups;

FIGS. 11A-C illustrate the structure of the initial hexameric aggregate of the PS.Ca.Pi complex obtain via molecular dynamic simulation as disclosed herein, FIG. 11A shows the aggregate of six PS.Ca.Pi complexes, FIG. 11B shows details of two of the head groups, FIG. 11C shows Ca—O bond distances (in nM) of an authentic PS.Ca.Pi complex as determined by RDF-EXAFS;

FIG. 12 is a TEM of a PS.Ca.Pi complex incubated in SCL for 15 min.;

FIGS. 13A-D include tube drawings of the inner structures of an Anx-H5 trimer (FIGS. 13A and 13B) and the outer contact points of Anx-H5 trimer including potential Pi and Ca²⁺ binding sites (FIGS. 13C and 13D);

FIGS. 14A and 14B illustrate molecular dynamic simulation as described herein of Anx-H5.PS.Ca²⁺ interactions including interaction of PS with the Anx-H5 domain 3 (FIG. 14A) and at the subunit interface between two monomer (domains 1 and 3) of the trimer (FIG. 14B);

FIG. 15 illustrates the effect of Anx-H5 on mineral formation by synthetic PS and PE-containing Ca.Pi complexes;

FIG. 16 illustrates the effect of Anx-H5 and Zn²⁺ on mineral formation by amorphous calcium phosphates;

FIGS. 17A and 17B illustrate segments of a molecular dynamic simulation as described herein in the process of determining Zn²⁺ binding sites on Anx-H5 both before (FIG. 17A) and after (FIG. 17B) Zn²⁺ binding;

FIG. 18 Illustrates the delay of onset of mineralization of Anx-H5 containing nucleators through incorporation of O-phospho-L-serine;

FIG. 19 illustrates a portion of a molecular dynamic simulation as disclosed herein including a sector of a 60 nm diameter MV showing the presence of Anx-H5 and associated Ca²⁺ and phosphate ions at the interface with a PS-rich membrane layer;

FIG. 20 illustrates the construction and identification of replication competent (RCAS) retroviral mediated gene delivery vehicle Anx-5 vectors;

FIGS. 21A-D illustrate phase-contrast micrographs of cultured chondrocytes including RCAS-green fluorescent protein (GFP) treated chondrocytes (FIG. 21A), control culture (FIG. 21B), chimeric RCAS-native Anx-5 transfected cultures (FIG. 21C), and chimeric RCAS-Anx-5 mutant transfected culture (FIG. 21D); and

FIGS. 22A and 22B illustrate intracellular [Ca²⁺] measurements on suspensions of cells released from two separate transfection experiments.

Repeat use of reference characters in the present specification and drawings is intended to represent the same or analogous features or elements of the present disclosure.

### DETAILED DESCRIPTION

It will be readily understood that the components of the presently disclosed subject matter, as generally described herein, could be arranged and designed in a wide variety of different configurations. Thus, it is to be understood by one of ordinary skill in the art that the present discussion is a description of exemplary embodiments only, and is not intended as limiting the broader aspects of the disclosed subject matter.

In general, disclosed herein are system and methods for development of compounds that can be utilized in one embodiment for preventing pathological calcification. Disclosed methods can also be utilized in development of compounds for treatment of other disease processes such as cancer, inflammation, Alzheimer's and viral budding. According to another embodiment, disclosed methods and systems can provide compounds for encouraging calcification, for instance in healing fracture callus. More specifically, systems and methods disclosed herein can provide a functional model that can accurately represent in great detail the first steps in protein-membrane interaction leading to calcification.

Compounds described herein can include those that interfere with pathologic calcification processes and thus may be used as part of a formulation or composition, for instance a topical dermal composition, a systemically administered composition, and the like, for preventing pathological calcifications, in osteoarthritis for example, and its associated conditions. Biologically inspired molecules that can interfere with pathological mineralization determined according to disclosed methods can be devoid of many of the deficiencies of existing therapies, for instance due to their lower toxicity and/or increased specificity to the pathology of interest.

Methods and systems as described herein can include in vitro and/or in silico dynamic molecular modeling of biological materials including representative mineralization models in conjunction with substances that can affect normal or pathogenic calcification processes. Specifically, the present disclosure relates to the inhibition or encouragement of formation of a quaternary complex consisting of calcium (Ca²⁺), inorganic phosphate (Pi), phosphatidylserine (PS) and annexin 5 protein (Anx-5), and in one particular embodiment, human annexin 5 protein (Anx-H5).

As disclosed in more detail herein, multiple aspects of the calcification nucleational complex and process have been reconstituted from the pure components and a working model has been developed that mimics the process of mineral formation induced by native MV. Disclosed modeling systems enable reproducible quantitative measurements of the effect of factors that alter the onset time, rate, length, and final quantity of mineral formed. Disclosed methods can enable systematic study of the effects of numerous factors thought to contribute to normal and/or pathological mineral formation. Through utilization of disclosed methods, implantable biomimetic materials can be constructed that can stimulate healing of recalcitrant bone defects. According to another embodiment, high throughput calcification assays and computer models can be utilized to screen for drugs that selectively inhibit de novo mineralization, but do not interfere with normal bone function. Compounds identified according to disclosed methods can be utilized in prevention of pathologies such as osteoarthritis and hardening of the arteries. Utilizing disclosed methods, lead compounds have been identified that can selectively inhibit de novo mineralization.

The physical and theoretical representations of disclosed systems have been built within a framework of physiological relevance, using accurate potential-energy functions refined to each system so that they can be tested in vitro and/or in silico as well as applied in vivo. Utilizing disclosed systems, high-throughput screening assays can be developed and harnessed to the powerful engine of computing chemistry to quickly and efficiently discover drugs that can enhance healing of bone fractures or prevent pathological calcification. Disclosed systems provide a route for construction of both empirical and theoretical models including ions, lipids and proteins by which calcification in vivo can be stimulated or inhibited for the treatment of dysfunctional calcification.

Furthermore, disclosed systems can be utilized to clarify the methodology of interaction between modeled cellular components and lipid bilayers in disease processes other than those directly involving calcification. Thus, disclosed methodology and understandings gained thereby can have important, broad reaching ramifications as well as application in additional biochemical areas such as oncology and virology. Accordingly, encompassed herein are methods, systems and compounds that can be utilized in applications in addition to the specific calcification applications described in detail.

For example, compounds determined according to disclosed methods can encourage the inhibition, blocking, and/or prevention of protein self-assembly and orientation on lipid bilayers in mammals. For instance, prophylactic administration of a phosphoserine, a phosphoethanolamine and/or their analogs, particularly when complexed with zinc (Zn²⁺) ions has been determined effective for such purposes through application of disclosed methods. Such small molecule analogs that interfere with the formation of the quaternary Anx-5.PS.Ca.Pi complex can also be used as potential anticancer, antiviral, anti-Alzheimer's and anti-inflammatory drugs as these disease processes can likewise involve Anx-H5/phospholipid membrane interactions. In addition, compounds developed according to disclosed systems and methods of action of the compounds that modify the level of expression of a key lipid, such as phosphatidylserine, and/or Anx-H5, or drugs that modulate their activities (i.e. via phosphorlyation, methylation, etc.) can be utilized as anti-arthritic, anticancer, antiviral and anti-inflammatory therapeutic agents.

Because of their importance to both normal and pathological calcification, disclosed systems and methods can in one embodiment model mechanisms involved in MV mineral deposition. Specifically, understanding gained regarding mechanisms of MV mineral deposition through application of disclosed systems can be of great value in the design of effective therapies for treatment of dysfunctional calcification.

Since their discovery 40 years ago, many studies have been directed toward analysis of MV composition. Biochemical analyses of isolated MV reveal that their lipid composition is quite different from that of the plasma membrane from which they derive. MVs are enriched in PS, sphingomyelin (SPH), cholesterol, and lysophospholipids, as well as various enzymes and porters including tissue nonspecific alkaline phosphatase, PHOSPHO1, matrix metalloproteases, phosphate transporters, etc., and have reduced amounts of phosphatidyicholine (PC). These differences indicate that during MV formation specific phospholipases are operative, as well as mechanisms for enriching PS. In the presence of Pi, Ca²⁺ binds to this lipid, forming ternary PS.Ca.Pi complexes that nucleate mineral formation.

During MV mineralization, membrane phospholipids become degraded in a manner characteristic of each lipid class. For instance, PS becomes rapidly complexed with newly forming mineral, and after an initial loss, breakdown of PS is reversed and net synthesis occurs. This unexpected Ca²⁺-dependent event results from a non-energy-dependent base-exchange in which serine displaces ethanolamine, the polar head group of phosphatidylethanolamine (PE), forming PS. This dramatic increase in PS occurs when MV calcification is most rapid. Other studies reveal that Vitamin D metabolites (e.g. 1α,25(OH)₂D₃) interact with receptors on MV membranes to activate phospholipase A2, producing lysophospholipids that further destabilize the MV membrane and matrix metalloproteases, which aid in the removal of inhibitory proteoglycans.

Accompanying membrane breakdown during cellular hypertrophy, PHOSPHO1, a phosphatase highly expressed in developing bone and MV that rapidly degrades phosphoethanolamine and phosphocholine released during membrane breakdown, contributes additional Pi for MV calcification. This selective degradation of MV lipids during induction of mineral formation appears to provide a mechanism for the exit of the newly forming mineral. Recent studies show that inhibition of this enzyme decreases the ability of MV to calcify. In addition, tissue non-specific alkaline phosphatase hydrolyzes residual organic phosphate compounds released by the dying cells, elevating levels of Pi in the extracellular fluid, as well as removing pyrophosphate, an inhibitor of mineralization.

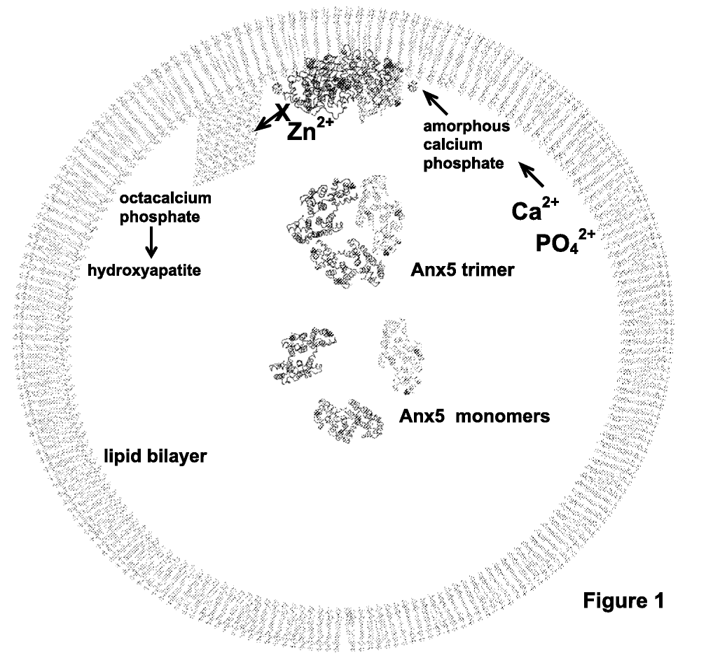

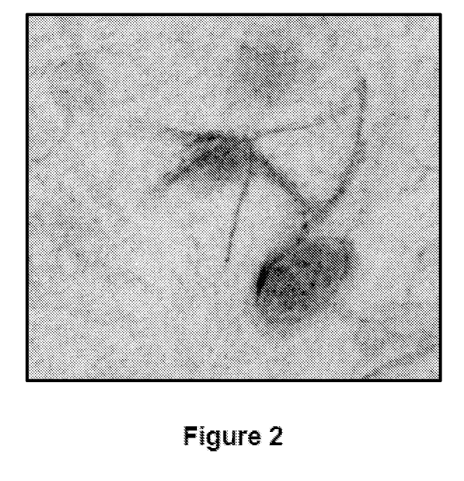

In vivo, and as schematically illustrated in FIG. 1, MVs load Ca²⁺ and Pi, enabling them to induce mineralization in conjunction with Anx-5 trimers at the interface with the phospholipid bilayer. FIG. 2 is an electron micrograph of MVs isolated from growth plate cartilage. As can be seen, the nucleational complex is located at the electron dense calcium phosphate deposits at the peripheral membrane of the MV. Newly formed mineral can also be seen growing from the MV into the matrix along collagen fibrils.

In vitro MV isolated form normal tissue also take up Ca²⁺ and Pi ions and form crystalline mineral when incubated in synthetic cartilage lymph (SCL). Studies of the kinetics and stoichiometry of Ca²⁺ and Pi accumulation reveal a consistent pattern. After an initial lag period of ˜1-2 hours, MVs rapidly accumulate large amounts of Ca²⁺ and Pi, forming an acid-phosphate-rich, OCP-like mineral as the first crystalline phase. MV mineral formation is strongly inhibited by Zn²⁺, but can be readily reactivated by chelating this metal ion. The ability of MVs to mineralize can also be destroyed by treatment with proteases, revealing that proteins are critical to their activity. However, this ability can be restored by treatment with detergents, which removes outer membrane lipids, but not the nucleational core of PS.Ca.Pi complex, ACP and annexins. But then, even brief exposure of this residual material to pH 6 isosmotic citrate buffer irreversibly destroys its ability to mineralize, causing major loss of Ca²⁺ and Pi. Finally, mineralization of MV is very sensitive to the pH of SCL; Ca²⁺ uptake is very slow at pH 7.2, is optimal at pH 7.6-7.8, but is totally blocked at pH 8, as is ACP. Mineral formation by PS.Ca.Pi complex is similarly pH sensitive. It is noteworthy that the pH of growth plate extracellular fluid is 7.6, the optimum for MV mineralization. Further elucidation of MV action, for instance via disclosed modeling systems, can lead to the development of biomimetic therapeutics, e.g. synthetic MV or nucleational complexes that directly induce mineralization and promote healing of bone fractures or recalcitrant bone injuries.

When nucleating agents such as MVs are introduced into synthetic lymph (SCL), numerous factors influence the timing of the onset of de novo mineral formation, as well as its rate, and the final quantity of mineral formed. Until recently, analysis of such parameters has been hampered by a lack of simple and reliable method for monitoring its progress. What was needed was a well-defined in vitro model that closely mimics in vivo mineralization and can be continuously monitored without disturbing the system. To study factors that regulate mineral formation, it is desirable to be able to define and measure their effect on different phases of this process, as well as the overall amount of mineral formation. Such a model should be reproducible and precise enough to enable accurate measurements of the effect of factors that regulate these aspects of mineral formation. To this end, disclosed model systems, which can incorporate one or both of in vitro and in silico modeling, has been developed that meets these criteria.

While MVs are associated with several proteins present in the extracellular matrix (e.g., proteoglycan link protein, hyaluronic acid-binding protein, and type II and X collagens), disclosed methods in one embodiment focus on the annexins and complexes they form with phospholipids, Ca²⁺ and Pi. The annexins belong to a super-family of PS-dependent Ca²⁺-binding proteins expressed in many tissues and species. In growth plate cartilage, it has been determined that annexins 2, 5, and 6 are the dominant proteins present in MV isolated from normal avian tissues. The presence of these annexins in MV isolated from articular chondrocytes and osteoblasts from a variety of species has also been confirmed.

The annexins are understood to be critical components of the mineralization machinery of normal MV. Key findings leading to this understanding include: 1) The two major EGTA-extractable MV proteins (30-36 kDa) bind to submicromolar levels of Ca²⁺ only in the presence of acidic phospholipids, such as PS, forming readily sedimentable complexes; and such properties are characteristic of the annexins. 2) Amino terminal and DNA sequence analyses have confirmed that these proteins are in fact annexins 2, 5, and 6. 3) Immunolocalization has revealed that the level of annexins is highest at the calcification front and is localized within MV. 4) Annexin 5 is found in MVs isolated from calcifying growth plate chondrocyte cultures, but not in non-mineralizing vesicles released into the culture medium. 5) Annexins contribute to the enrichment in PS and the large amount of complexed Ca²⁺ present in MV. 6) Annexin 5 binds to native type II and X collagens, promoting interaction between MV and the extracellular matrix, enhancing mineral formation. 7) Patch-clamp electrophysiology studies have indicated that annexin 5 interacts with acidic phospholipid bilayers to form voltage-gated channels with high selectivity for Ca²⁺. 8) Expression of annexin 5 is upregulated during GP development and it is a dominant protein in calcifying chondrocytes and MV. 9) Finally, annexin 5 markedly enhances the nucleational activity of the PS.Ca.Pi complex.

Annexin 5 is abundantly present in extracellular granular structures akin to MV during development of osteoarthritis (OA). MVs are also seen with mineral deposits in regions of late-stage human OA cartilage. Expression of annexin 5 is significantly upregulated at both the message and protein level in the entire depth of osteoarthritic cartilage suggesting a link to this disease. In addition, it has been reported that OA chondrocytes undergo terminal differentiation, releasing annexin 5-containing MV. Accordingly, because of its activation of the PS.Ca.Pi complex, annexin 5 can be included in a modeling system as disclosed herein as it is a promising target for the design of therapies to reduce or prevent pathological calcification.

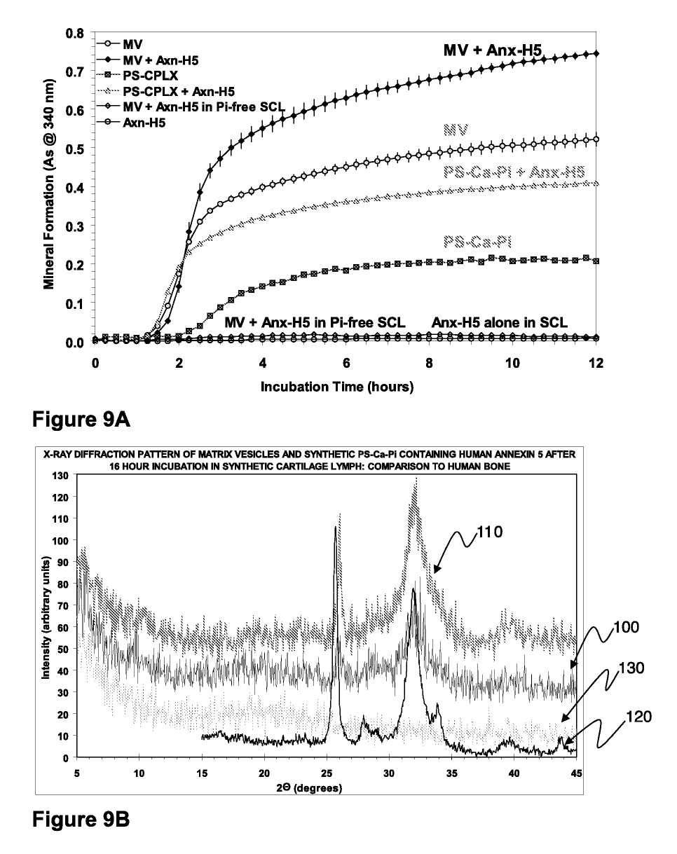

Disclosed modeling systems can incorporate Anx-5 as monomers or as the formed trimer. In solution, annexin 5 is monomeric, but upon Ca²⁺-dependent binding to planar PS—PC monolayers or supported bilayers, Anx-5 forms highly ordered two-dimensional (2-D) crystalline arrays. Two main types of annexin arrays have been described—with either p6 or p3 symmetry. Both types are derived from a building unit composed of a trimer of annexin 5 monomers. The annexin 5 trimer is also the building unit of 3-D annexin crystals. The p6 form occurs when the PS content is low (5-20%); the p3 form occurs when the PS level is high (>40%). AFM studies indicate that the p6 type forms first, but the two forms are reversible and stable. When annexin 5 binds to PS.Ca.Pi, it stabilizes the planar arrays (see, e.g., FIG. 9). Studies further indicate that annexin 5 forms 2-D crystalline arrays that facilitate and stimulate growth of crystalline Ca.Pi mineral once nucleation has occurred. Comparison of the lattice parameters of the basic p6 crystal form with those of OCP or HAP indicates that nine of the hexameric clusters of PO₄³⁻ and Ca²⁺ ions in their unit cells could potentially fit in the space between the annexin 5 trimers. This arrangement can explain why annexin 5 stimulates crystalline mineral formation from PS.Ca.Pi. In disclosed computer simulation studies, discussed further below, interaction of PS with Pi and Ca²⁺ has been explored using molecular dynamics to elucidate the mechanism of PS.Ca.Pi formation as well as the packing arrangement of Ca²⁺, and PO₄³⁻ after they interact with specific functional groups of PS and with each other. Disclosed in silico and in vitro modeling systems demonstrate that annexin 5 is directly involved in critical early steps of MV mineralization by enhancing the activity of this PS.Ca.Pi complex.

Disclosed modeling systems can also incorporate one or more electrolytes believed or understood to be important to neocalcification. The ability to isolate MV from normal growth plate (GP) tissues has enabled the analysis of electrolyte components. Direct chemical analysis, and slam-freeze transmission electron microscopy (TEM) methods have shown that normal MV contain high levels of Ca²⁺ and Pi, mainly in an insoluble form, complexed in part with PS and MV annexins. A wide variety of physical methods have shown that the initial mineral in MV is not crystalline. Using solid-state ³¹P-NMR methods, the principal components of this solid phase have been shown to be the PS.Ca.Pi complex and amorphous calcium phosphate (ACP). MVs also contain significant amounts of Na⁺, K⁺, Mg²⁺, and Zn²⁺. Mg²⁺ stabilizes ACP and reduces the nucleational activity of PS.Ca.Pi complex. Recent work shows that annexin 5 not only overcomes Mg²⁺ inhibition, but significantly potentiates mineral formation by the complex. Zn²⁺, however, inhibits the Ca²⁺ channel activity of annexin 5; and at lower levels Zn²⁺ also stabilizes octacalcium phosphate (OCP) in MV slowing its conversion to hydroxyapatite (HAP). Thus, both Mg²⁺ and Zn²⁺ are important regulators of MV mineralization. Other divalent cations can be included in disclosed modeling systems. For instance, nickel, cobalt, and/or copper ions, which are understood to promote binding of annexin-5 to phospholipids can be incorporated into disclosed models and examined for utilization in encouraging calcification, for instance in healing fracture callus.

Additional components of the nucleation core of MV can be incorporated in disclosed models. The discovery of the nucleation core stemmed from the early finding that acidic phospholipids were complexed with Ca²⁺ at sites of early mineralization. Accordingly, one or more phospholipids can be incorporated in the disclosed models. While PS can be incorporated in one embodiment, the present disclosure is not limited to this phospholipid. Disclosed models can incorporate any phospholipid or combination of lipids. For instance, a model can include other common biological phospholipids such as phosphatidic acid, phosphatidyl inisitol, cardiolipin, and the like. When incorporating one or more phospholipids in a model, the phospholipids can be incorporated in a random formation or in an organized fashion. For instance, in one embodiment, a model can include phospholipids as an organized phospholipid bilayer. Such an embodiment can be beneficial when examining certain aspects of mineralization such as, for example, the calcium dependent formation of ordered annexin structures, such as the annexin trimer, and the lipid membrane surface.

In one embodiment, a model can focus on formation of PS.Ca.Pi complexes. PS.Ca.Pi complexes have been found at the initial stages of almost all calcifying tissues: growth plate cartilage, tumors, bone, and MV. PS.Ca.Pi and associated proteolipids act as nucleators of HAP formation. PS is largely confined to the inner MV membrane where electron micrographs show electron-dense calcium phosphate deposits. Synthetic Mg²⁺-free PS.Ca.Pi complexes are powerful nucleators that rapidly induce HAP formation when incubated in SCL; Mg²⁺-containing PS.Ca.Pi complexes, in contrast, are weak nucleators. Annexin 5, the dominant lipid-dependent Ca²⁺-binding protein of MV, is believed to activate the nucleational activity of Mg²⁺-containing PS.Ca.Pi.

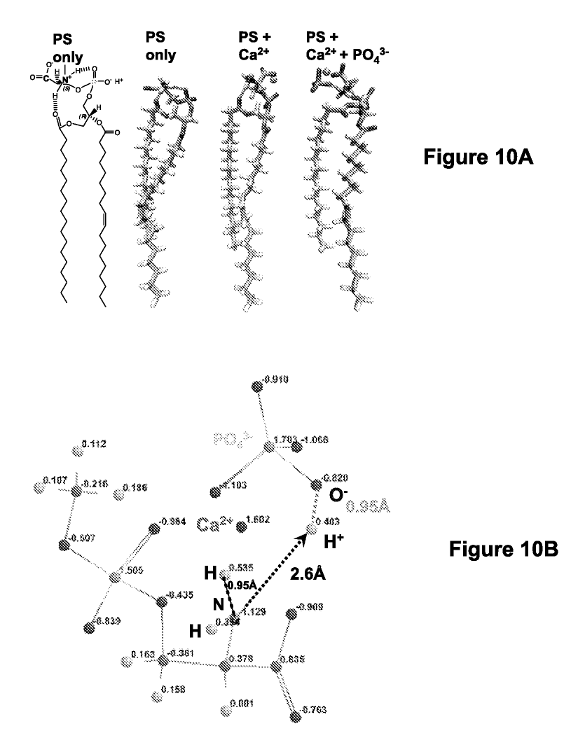

Insight into how the pure PS.Ca.Pi complex induces crystalline mineral formation has come from high resolution TEM studies of the complex incubated briefly in SCL. Apparent are very thin sheet-like “quasi-crystals”, which at high magnification resolve into arrays of electron-dense dots 7.5-8.0 Å in diameter as illustrated in FIG. 10. These appear to be ion clusters at the polar head group of PS, arranged in quasicrystalline order. As can be seen, periodically, there are small diamond-shaped areas where two layers, evident as double electron density, show more clearly ordered linear arrays. These are interpreted to be sites of epitaxy for the incipient formation of OCP-like crystals that FT-IR studies reveal occur during initial MV mineralization. The speed of this nucleation phenomenon is due to the thermal mobility of the individual PS.Ca.Pi units in the monolayer, which enable rapid formation of more ordered epitaxial sites.

In general, a modeling system can include Pi among the biological components. For instance, when modeling the formation of PS.Ca.Pi, Pi will be present with the lipid before introduction of Ca²⁺, as this is believed to be an important aspect of mineralization. This feature also occurs in cells that form MV, where high levels of Pi are present and intracellular levels of Ca²⁺ are low until MV formation is in progress. Addition of Ca²⁺ to Pi-rich solutions can cause rapid formation of ACP, an ephemeral mineral that rapidly and spontaneously converts to HAP unless stabilized by various agents, such as Mg²⁺, certain proteins (e.g. casein), and acidic lipids such as PS. Interaction of PS during formation of ACP leads to production of PS.Ca.Pi.

According to one embodiment of the disclosed subject matter, a dynamic molecular model can be designed incorporating one or more of the biological components discussed above as well as additional biological components believed or known to be involved in any aspect of neocalcification such as common biological ions such as sodium and carbonate. A model can accurately and specifically examine the interaction of the selected components, for instance during a mineralization/calcification process. Moreover, a model can incorporate one or more additional components for examination of specific effects of the additional components on a normal interaction process. These additional components can then be further examined, for instance according to in vitro assay processes so as to determine their usefulness as, e.g., calcification inhibitors.

In general, a modeling system as disclosed herein can incorporate in silico and/or in vitro modeling. For example, in one embodiment, substances suspected of affecting calcification can be quickly and efficiently screened through in silico modeling, with in vitro examination then carried out on substance candidates identified through the in silico processes. Thus, disclosed methods and systems can save a great deal of time in drug development and the like. More specifically, materials as discussed above that are integral to calcification, can be examined for in silico and in vitro systems that accurately model in vivo systems so as to develop compounds that can affect calcification.

In order to accurately model calcification processes in silico, accurate parameters and characteristics of the materials to be incorporated into a model should be determined and integrated into the system. For instance, using NAMD (NAno-scale Molecular Dynamics) software (Theoretical Biophysics Group and the National Institutes of Health Resource for Macromolecular Modeling and Bioinformatics, Beckman Institute, University of Illinois, Urbana-Champaign, Ill.) can allow application of molecular mechanics to study the formation and the progressive changes in structure of the PS.Ca.Pi complex over time upon development and incorporation of accurate characterizing information of the components to the software. Brief reviews on potential energy functions used in MD simulations are known in the art, any of which can be incorporated into disclosed modeling systems. For example, properties that can be incorporated can include, without limitation, lipid head area, stability, diffusion, radial distribution functions, structures and atomic distances throughout the simulation trajectory. In addition, Gaussian 3.0 models of OCP and HAP can be built and visualized to understand changes that must occur in the packing arrangement of the individual PS.Ca.Pi monomers in the two-dimensional space of the lipid monolayer to enable them to nucleate either of these crystal forms. Together, this information can teach the initial steps involved in Ca²⁺ and Pi accumulation during MV mineralization and in developing structure-based drugs targeted to inhibit these very first steps of nucleation.

In one embodiment, a system can explore the mechanism of PS interaction with Ca²⁺ and Pi. Specifically, molecular dynamic (MD) simulation can be utilized to gain insight into the atomistic mechanism by which PS.Ca.Pi complex can form. In general, a model can include PS, as PS-based Ca²⁺.Pi complexes have previously been associated with normal and pathological calcification in vivo. For example, a nanoscale domain consisting of PS, Ca²⁺, PO₄³⁻, K⁺ and H₂O, can be constructed and visualized.

There are several software programs available for purchase or free download as may be utilized in development of disclosed modeling systems. For instance, Tool Command Language scripts run from within Visual Molecular Dynamics (VMD) software can be utilized. Other visualization programs as may be utilized include, without limitation, Hyperchem 8.0, available from Hypercube, Inc.; Scigress Explorer (previously CaChe) available from Fujitsu, Inc.; Spartan '06, available from Wavefunction, Inc.; and INSIGHT II available from Accelyris, Inc. Using the software, an assembly of PS molecules can be created, orienting the lipid tails approximately coincident with the z-axis of the simulation cell. The PS can be arranged as found in vivo, for instance with the phosphodiester phosphate atoms of the head group approximately 8 Å apart in a loose hexagonal arrangement 4 Å below the x-y plane. The basic unit of ACP can be incorporated in a simulation. For instance, Ca²⁺ and PO₄³⁻ atoms can added in a random arrangement 4-7 Å above the PS head groups. Next, since PS.Ca.Pi complexes are prepared in KCl containing solutions, to neutralize the negative charge of PS, K⁺ atoms can be added to the ensemble. Finally, the system can be hydrated by adding bulk H₂O above the hydrophobic region of PS. The number of each type of material included in the ensemble can vary with regard to processing system as well as with regard to the relative amounts of each compound found in vivo. For instance, a complete system can include anywhere from a few hundred up to several thousand atoms, as desired.

To perform MD simulations, a force field for the molecules of interest is required. One commonly used force field as may be used is CHARMM (Chemistry at HARvard Macromolecular Mechanics), AMBER (Assisted Model Building and Energy Refinement), GROMACS, the force field optimized for the package of the same name, and the OPLS (Optimized Potential for Liquid Simulations) force field, developed by William L. Jorgensen at Purdue University and later at Yale University. Available force fields contain parameters for many molecules of interest in disclosed systems. Parameters for molecules not available can be created through quantum chemistry methods. For instance, CHARMM contains parameters for a growing list of lipid molecules; however, PS is not among them. Therefore, when utilizing a CHARMM force field, parameters for PS can be created. According to one method, parameters for a molecule can be created by starting from basic fragments of similar molecules already present in the force field. For example, using parameters for the paimitoyl- and oleoyl-chains, and the glycerol backbone of phosphatidylethanolamine (PE), and similar molecular fragments of serine and palmitate, available in CHARMM, initial parameters for PS can be compiled. The partial charges of atoms of the molecule, for instance the PS head group were can then be further refined using ab initio quantum chemical calculations, for instance using Gaussian 03W (49) (Gaussian, Inc., Wallingford, Conn. 06492) and Trident 1.0.0 (Wavefunction, Inc., Irvine, Calif. 92612) software using a model compound. By way of example, using O-methyl-L-serine phosphate anion as the model compound, its geometry optimized in water, when determining the partial charges of a PS head group. Basis sets can be optimized using a stepping-stone approach in which the geometries can be sequentially optimized, for instance using the Hartree-FockF/3-21G and Hartree-Fock/6-31G* basis sets. Thus, accurate partial charges of atoms in a molecule, for instance the PS head group atoms, can be determined and added to the basic topology library.

Additional parameters such as non-degenerate bond, angle, dihedral potential parameters, long range interactions including Coulomb and Lennard Jones two-body interaction terms, and so forth for each molecule can be obtained or calculated from other sources as are generally known to one of skill in the art, and added to the topology library.

For instance, in one embodiment, a dihedral potential can be calculated through optimization of dihedral potential for the simplest possible molecule and then apply it to larger ones containing the same dihedral. In another embodiment, a dihedral potential can be calculation through optimization of the dihedral parameters to best describe a large number of different molecules. In both embodiments, the dihedral parameters can be computed from ab initio methods as are known in the art including initially, perform ab initio calculations that include scanning dihedral (or improper) of interest, optimizing the geometry at each step, and calculating the change in potential energy. For this purpose, either perturbation (MP2), restricted Hartree-Fock (RHF) or hybrid methods between Hartree-Fock and density functional theory (B3LYP) can be used. The basis sets chosen for the geometry optimization are generally at least 6-31 g and may go up to 6-311 g**, depending on the size of the molecule in question. Following ab initio calculations, the dihedral parameters are set to zero and the potential energy of each optimized configuration are computed (note that this requires the knowledge of all other force field parameters in the molecule.

Similarly, parameters of other atoms and/or molecules in a simulation can be refined and added to a file. For example, the non-bonded interaction parameter for Ca²⁺ in the presence of PO₄³⁻ can be refined to be more representative of the properties of a system including both materials based on the methods of Marchand and Roux (Proteins. 1998 Nov. 1; 33(2):265-84) and Zahn (Anorg. Allg. Chem. 630:1507-1511, 2004).

Favorable electrostatic and Van der Waals interactions can be incorporated into a simulation. These interactions can be calculated according to known practices, for instance based on method of Aqvist (Aqvist, J. Ion-Water Interaction Potentials Derived from Free Energy Perturbation Simulations. J. Phys. Chem. 94, 8021, (1990)). For example, in order to determine the parameters rij* (the internuclear separation of the ij pair at the potential minimum) and e, the potential well depth for the ion at this minimum value, the following method can be utilized:
Given is the Lennard-Jones potential:

U(r)=e(r*/r)¹²−2e(r*/r)⁶ [1]
which is commonly rewritten for the ij pair of ions as:

U(rᵢⱼ)=(AᵢAⱼ/rᵢⱼ¹²)−(BᵢBⱼ/rᵢⱼ⁶) [2]
where the A and B parameters are given in Aqvist. To find rij*, we take the derivative of [2] with respect to rij:

dU(rᵢⱼ)/drᵢⱼ=−12AᵢAⱼrᵢⱼ⁻¹³+6BᵢBⱼrᵢⱼ⁻⁷ [3]
and set the right side of [3] to zero:

−12AᵢAⱼrᵢⱼ⁻¹³+6BᵢBⱼrᵢⱼ⁻⁷=0 [4]

then solve for rij at this minimum which is what we call rij*:

rij*=(2AᵢAⱼ/BᵢBⱼ)^{1/6} [5]
Using [5] in concert with Aqvist's paper, we can proceed to determine rij* for Mg²⁺, Ca²⁺, Sr²⁺, and Ba²⁺ (the monovalent species are already in the AMBER parameter set) where species i is one of the cations and species j is the oxygen in water. As an example, rij* for Ca²⁺ is calculated.

From Aqvist, it is known that ACa²⁺=264.1 and BCa²⁺=18.82. The water model used in AMBER is TIP3P [W. L. Jorgensen, J. Chandrasekhar, J. D. Madura, R. W. Impey, and M. L. Klein, J. Chem. Phys. 79, 926 (1983)]; the cation interacts with the oxygen in water, thus we need the A and B parameters for the oxygen in TIP3P water (AO=762.89 and BO=24.39).

r_{O—Ca}²⁺*=(2×762.89×264.1/24.39×18.82)^{1/6}=3.09437 Å [6]
In [6], r_{O—Ca}²⁺* is the sum of r_{O}* and r_{Ca}²⁺*:

r_{O—Ca}²⁺*=r_{O}*+r_{Ca}²⁺* [7]
Rearranging [7], and using r_{O}*=1.768 Å from Aqvist (note that this number includes the hydrogens implicitly):

r_{Ca}²⁺*=r_{O—Ca}²⁺*−r_{O}*=3.09437−1.768=1.3264 Å [8]
For e, comparing [1] to [2]: It is clear that

AᵢAⱼ=eᵢⱼ(rᵢⱼ*)¹² [9]

and

BᵢBⱼ=2eᵢⱼ(rᵢⱼ*)⁶ [10]
Since i=j for each species, the subscripts have been dropped in the following discussion. The square of [10] is:

B⁴=4e²(r*)¹² [11]
and [11] can then be solved for (r*)12:

(r*)¹²=B⁴/4e² [12]
Rearrange [9] to

(r*)=A²/e [13]
[12] to [13] can then be equated to solve for e:

e=B⁴/4A² [14]
Returning to the Ca²⁺ example, recall from Aqvist we have ACa²⁺=264.1 and BCa²⁺=18.82. Plugging these quantities into [14]:

e(Ca²⁺)=18.824/4×264.12=0.44966 kcal/mol

These values can then be put into the parameter file that NAMD uses to do calculations during the simulation. For example, a file that includes these data can be generated. For instance, a file can be named frcmod_Ca.in—and set up as follows (there are a total of 6 lines in the file, including the blank line):

\# First line; parameters for Ca++.

MASS

CA 40.08

NONB

CA 1.3264 0.44966 (adjusted, from Aqvist)

In tLEaP, for example, the following would then be incorporated:

>loadamberparams frcmod_Ca.in

Loading parameters: ./frcmod_Ca.in

Reading force field mod type file (frcmod)

>

and so on.

Upon development of a topology library for all components of a system to be modeled, molecular dynamic simulations can be run on a suitable processing machine. A preferred system for a specific simulation will generally depend upon the size of the system, i.e., number of atoms in the system, as well as the time period over which the simulation is to be run. For instance, a model including up to about 100,000 atoms run over a simulation period of up to about 10 nanoseconds can generally be run on a desktop size machine, for instance a quad-core dual-5150 Xeon processor machine. Larger simulations can utilize more powerful systems, for instance CUDA (Computer Unified Device Architecture) systems (available from Nvidia Corporation of Santa Clara, Calif.), or machines such as Cray XT3 machine supercomputing machines available at sites such as the Pittsburgh Supercomputing Center, can be utilized for larger simulations, for instance those including more than about 50,000 atoms and/or those run for a longer simulation period, for instance up to about 1 millisecond. Of course, the size of any simulation can be increase depending upon the capabilities of the system. For instance, million atom simulations can be run on supercomputers, and almost millisecond simulations have been run on larger computers.

Other parameters of a simulation can be added in various levels of complexity. For example, a system can be minimized to remove unfavorable van der Waals contacts, for instance by using the conjugate gradient algorithm of NAMD for 100 steps; bond lengths can be fixed for all TIP3-explicit water molecules, short-range non-bonded interactions can be cut off smoothly, for instance between about 12 and about 15 Å, and so forth. Other physical characteristics of a simulation can also be incorporated into the model, for instance, a system can be warmed and equilibrated at 310 K using a 2.0 fs time step. The structure can then be subjected to MD simulations.

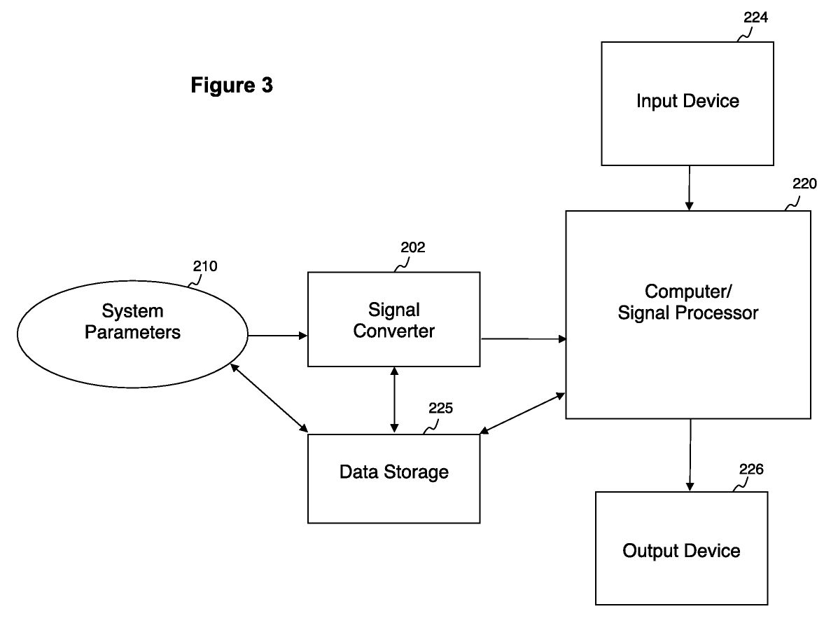

More particular aspects of how a subject dynamic in silico modeling system can be implemented will now be discussed with reference to FIG. 3. The system input parameters for all materials to be included in a model can be provided to a computer 220 or other general-purpose or customized computing or processing device having any suitable form of hardware architecture or configuration that can be adapted to implement digital signal processing techniques. Although FIG. 3 only illustrates a single computer or processing device 220, it should be appreciated that multiple processors operating independently or in a collaborative series, parallel or distributed fashion may be utilized to implement the subject technology. Embodiments of the methods and systems set forth herein may be implemented by one or more of such computers 220 that access software instructions rendered in a computer-readable form, which thus configure the computing device(s) to function as special purpose machine(s) adapted to perform designated algorithmic steps. Software instructions may be stored in one or more portions of computer-readable media as computer-readable instructions which, when executed by at least one computer 220, cause the at least one computer to implement one or more embodiments of the methods disclosed herein. Any suitable computer-readable medium or media may be used to implement or practice the presently-disclosed subject matter, including diskettes, drives, and other magnetic-based storage media, optical storage media, including disks (including CD-ROMS, DVD-ROMS, and variants thereof), flash, RAM, ROM, and other memory devices, and the like. Further, any suitable programming, scripting, or other type of language or combinations of languages may be used to implement the teachings contained herein. Embodiments of the methods and systems set forth herein may alternatively be implemented by hard-wired logic or other circuitry, including, but not limited to application-specific circuits. Of course, combinations of computer-executed software and hard-wired logic may be suitable, as well.

In one embodiment, the computer-readable media is embodied by data storage element 225 depicted in FIG. 3. Data storage 225 may be coupled to computer/signal processor 220 such that the processor 220 can have direct access to executable instructions or to system data which may be stored therein. Additional data that may be stored in data storage 225 may include one or more of the system parameters (i.e., specific molecular parameters provided at element 210), threshold levels customizably selected by a user via input device 224, and processing outputs generated by the processor 220, including but not limited to data indicating the molecular interaction of specific materials incorporated into the system as described herein.

Referring still to FIG. 3, system signals 210 may in some embodiments be relayed through a signal converter 202 before being received at computer 220. Signal converter 202 may be employed to render system signals 210 in a format that is compatible with the computer 220. In one example, signal converter 202 corresponds to an analog-to-digital (A/D) converter or the like. One or more input device 224 may also be coupled to computer 220. Input device may correspond, for example, to such devices as a keyboard, mouse, touch-screen, scanner, microphone or other device with which a user may provide input to computer 220. Input device 224 may be employed by a user to set customizable threshold limits and other parameters associated with the subject system diagnostics. Output device 226 may correspond, for example, to one or more of such devices as a display, monitor, printer, speaker or the like for providing output to a user. A visual, audio or other output preferably relays in a user-accessible form the results of the system analysis conducted by computer 220. In one embodiment, numeric and/or graphical illustrations of the comparisons may be provided. Variations to the exemplary input and output devices should be appreciated by one of skill in the art and is not intended to limit the scope of the present technology. It should be appreciated that in some embodiments, a single device can serve as an integrated input and output (I/O) device as opposed to having separate devices 224 and 226 as illustrated in FIG. 3.

The described molecular dynamic (MD) in silico simulations provide direct visual imaging of the atomic nature of the PS.Ca.Pi complex that aid in design of therapeutic agents to prevent such interactions.

In conjunction with or independently of in silico modeling systems, disclosed subject matter also encompasses in vitro systems for examining neocalcification components. For example, disclosed systems can include in vitro studies of annexin proteins in conjunction with MV as well as individual MV components including, but not limited to PS, Ca²⁺, Pi, and so forth. For example, following initial in silico modeling of a system to examine the effects of a potential calcification inhibitor, the candidate inhibitor can then be tested in an in vitro setting, for instance in a chondrocyte culture, to confirm the viability of the potential calcification inhibitor for further testing, e.g., in vivo testing. Of course, any suitable in vitro testing methodology as is known in the art can be utilized to determine the effect of examined materials on a calcification system, as will be apparent to one of skill in the art. Thus, disclosed methods and systems can be utilized to efficiently screen a plurality of potential compounds for their effect on calcification.

Compounds discovered through utilization of disclosed modeling systems and methods can include ions, small molecules, peptides, proteins, and the like. For example, as described in further detail in the Example section, below, disclosed methods and systems have been used to illustrate the effect of zinc ion on a calcification system. For instance, in the presence of zinc ion, the annexin 5 trimer can still form, but the trimer is formed with a distinctly different arrangement, which in turn effects the interaction of the annexin 5 trimer with a phospholipid membrane and the opening of calcium channels through the membrane. Hence, through alteration in the formation of the Anx-5 trimer in the presence of zinc, neocalcification can be inhibited through prophylactic administration of zinc to a potential calcification site in vivo.

Other materials of interest can be examined for effect during other steps of neo calcification including, without limitation, effect on formation of the PS.Ca.Pi complex, effect on formation of Anx-5, effect on interaction between Anx-5 and the phospholipid bilayer, effect on interaction between the PS.Ca.Pi complex and Anx-5, and so forth. For example, low molecular analogs of phospholipids, and in one particular embodiment, low molecular analogues of PS, have been examined and found to operate as a competitive inhibitor of PS binding Anx-5, and found to delay mineralization induction in model systems. One of the applications of the disclosed subject matter is the development of effective, low-cost therapies for the treatment of pathological calcifications such as osteoarthritis. This can be achieved by using in vitro and/or in silico models as described above to screen for drug candidates that can interfere with the very first steps of de novo mineral formation, for example, interfering with Anx-H5 trimer assembly. For instance, O-phospho-L-serine, and its derivatives are encompassed herein—low molecular weight, e.g., usually about 1000 Daltons or less—analogs of PS.

The chemical structure of O-phospho-L-serine is shown below:

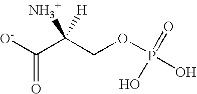

Since phosphatases such as TN-ALP and PHOSPHO1 are enriched in MV and other sites of mineralization, and might readily degrade phosphoserine, chemical modification of its structure can be carried out on the compound to impart resistance to degradation. One approach is to use a phosphono derivative that would be inert to these phosphatases, such as 2-amino-4-phosphonobutyric acid

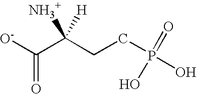

Further modifications, such as esterification, can be used to facilitate membrane transport of the molecule, an example of which is shown below:

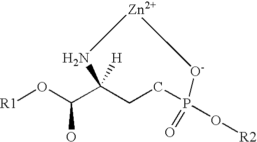

where
R1, R2 are independently H, CH₃, or a substituted or unsubstituted acyl chain up to about 20 carbons in length.

Once inside the cell, this pro-drug version can be activated by ubiquitous esterases in mammalian cells, cleaving the ethyl groups and reforming the charged species. Delivered as the salt, for instance the Zn²⁺ salt as shown, this molecule can effectively inhibit several targets at the earliest stages of mineralization: at Anx-H5 trimer formation, at TN-ALP or at PHOSPHO-1 preventing release of Pi, and at Anx-H5-stimulated PS.Ca.Pi nucleation of HA, thus having synergistic effects.

Other low molecular weight analogs encompassed herein include, without limitation:

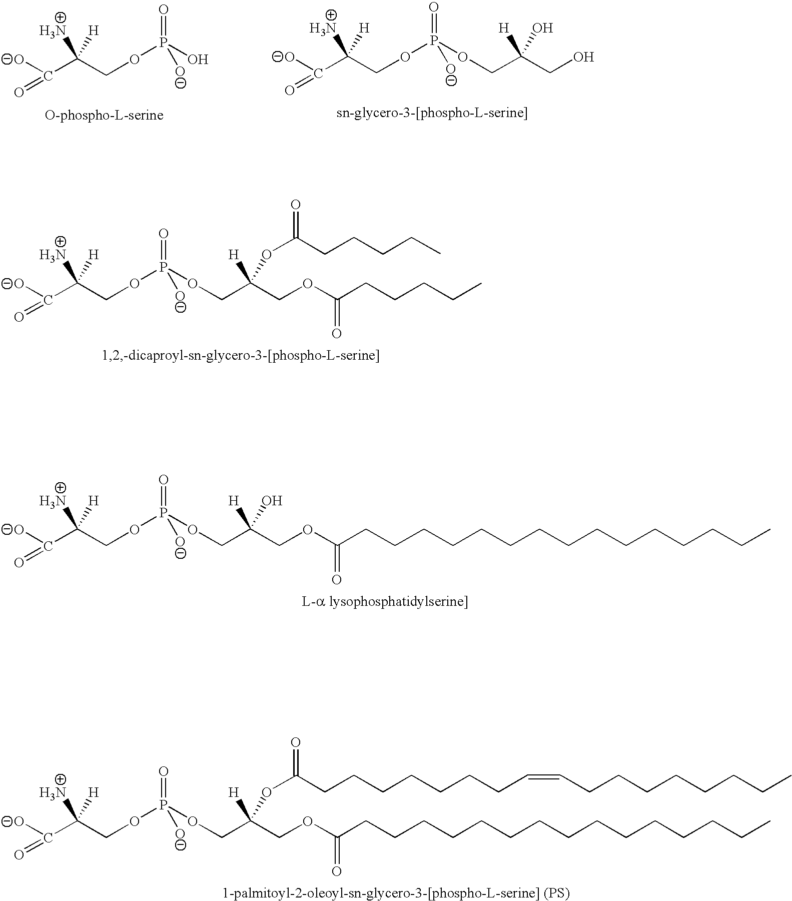

Other zinc complexes are also encompassed by the disclosed subject matter. For instance, zinc-pyrithione complex is another example of a zinc-small organic molecule complex can bind PS and interfere with de novo mineral formation. Such compounds, e.g., small molecule compounds, salts of the active compounds, and the like, can be utilized as topical applications in one embodiment. For instance, in a topical anti-osteoarthiric topical application, in one embodiment.

Other representative compounds can include variations of components of the system, for instance, annexin and PS compounds that include variations that interfere with calcification. For example, through utilization of disclosed modeling systems, specific mutations to the Anx-5 chain can be examined in detail with regard to effect on calcification formation as well as with regard to effect on other biological materials of a system. Thus, prior to any in vivo utilization, a better understanding of the full effects of a compound can be determined, saving both time and money in a screening method.

Other compounds that can effect initial mineralization can include analogues of lipids other than PS, cations such as magnesium (Mg²⁺), low molecular weight phosphate analogues such as vanadate, arsenate, small molecules such as phosphonoformic acid and phosphonoacetic acid and so forth.

Compounds obtained through utilization of disclosed methods, for instance calcification inhibitors such as compounds incorporating zinc or magnesium and low molecular analogs of PS as discussed further herein, or compounds to encourage calcification initiation, such as compounds including nickel, cobalt, copper or other divalent cations that promote binding of annexin to a phospholipid, can be provided to a subject in need thereof in pharmaceutically acceptable formulations using formulation methods known to those of ordinary skill in the art. These formulations can generally be administered by standard routes. For example, the formulations may be administered in one embodiment directly to a calcification location, for instance through direct application or via direct injection of a formulation to the targeted calcification site, e.g., a cartilaginous or synovial joint, a vascular site, or the like. In other embodiments, however, the formulations may be administered indirectly to the targeted tissue for instance as a medicament in a gauze or a salve, topical application near the affected site, and the like.

The formulations can be delivered intravenously in a systemic delivery protocol. In situ polymerizable hydrogels, as are generally known to those of skill in the art, and discussed further below, are another example of a delivery vehicle that can be utilized in a delivery protocol, for instance in an intravenous delivery directly to a targeted joint.

Formulations including disclosed compounds can include additional agents. Such agents can be active agents, providing direct benefit to a targeted tissue, or may be supporting agents, improving delivery, compatibility, or reactivity of other agents in the composition.

A formulation can include one or more buffers as are generally known in the art. For example, a formulation can be formulated with inclusion of a biocompatible buffer such as distilled water, saline, phosphate buffers, borate buffers, HEPES, PIPES, and MOPSO. In one embodiment, a formulation can have a pH of between about 5.5 and about 7.4.

Formulations for parenteral delivery, e.g., via injection, can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. In addition, the composition can contain minor amounts of auxiliary substances such as wetting or emulsifying agents, pH buffering agents and the like that can enhance the effectiveness of the active compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents.

Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like.

In one embodiment a delivery method can include use of timed release or sustained release delivery systems as are generally known in the art. Such systems can be desirable, for instance, in situations where long term delivery of the agents to a particular location is desired. According to this particular embodiment, a sustained-release matrix can include a matrix made of materials, usually polymers, which are degradable by enzymatic or acid/base hydrolysis or by dissolution. Once located at or near the target tissue, e.g., inserted into the body, for instance in the form of a patch or a stent such are generally known in the art, such a matrix can be acted upon by enzymes and body fluids. The sustained-release matrix can be chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone.

When an effective amount of a compound is administered by intravenous or subcutaneous injection, the compositions can generally be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection can contain, in addition to a compound as disclosed herein, an isotonic vehicle such as Sodium Chloride Injection, Ringer's Injection, Dextrose Injection, Dextrose and Sodium Chloride Injection, Lactated Ringer's Injection, or other vehicle as known in the art. The treatment composition of the present invention may also contain stabilizers, preservatives, antioxidants, or other additives known to those of skill in the art.

The dosage of a compound as disclosed herein can depend on the disease state or particular condition being treated and other clinical factors such as weight and condition of the human or animal and the route of administration of the compound. The disclosed compounds can be administered between several times per day to a single treatment protocol. Optionally, treatment agents could be delivered according to the disclosed process either acutely, during a one-time intervention, or chronically, for instance using multiple administrations or optionally a single administration of a timed or sustained releases system. It is to be understood that the present disclosure has application for both human and veterinary use. The methods of the present invention contemplate single as well as multiple administrations, given either simultaneously or over an extended period of time. In addition, disclosed compounds can be administered in conjunction with other forms of therapy, e.g., surgical repair of a synovial joint.

In another embodiment, a compound can be targeted by use of a hydrogel delivery vehicle. Hydrogels are herein defined to include polymeric matrices that can be highly hydrated while maintaining structural stability. Suitable hydrogel matrices can include un-crosslinked and crosslinked hydrogels. In addition, crosslinked hydrogel delivery vehicles of the invention can optionally include hydrolyzable portions, such that the matrix can be degradable when utilized in an aqueous environment, e.g., in vivo. For example, the delivery vehicle can include a cross-linked hydrogel including a hydrolyzable cross-linking agent, such as polylactic acid, and can be degradable in vivo.

Hydrogel delivery vehicles can include natural polymers such as glycosaminoglycans, polysaccharides, proteins, and the like, as well as synthetic polymers, as are generally known in the art. A non-limiting list of hydrophilic polymeric materials that can be utilized in forming hydrogels of the present invention can include dextran, hyaluronic acid, chitin, heparin, collagen, elastin, keratin, albumin, polymers and copolymers of lactic acid, glycolic acid, carboxymethyl cellulose, polyacrylates, polymethacrylates, epoxides, silicones, polyols such as polypropylene glycol, polyvinyl alcohol and polyethylene glycol and their derivatives, alginates such as sodium alginate or crosslinked alginate gum, polycaprolactone, polyanhydride, pectin, gelatin, crosslinked proteins peptides and polysaccharides, and the like.

Subject matter encompassed in the present disclosure can be better understood with reference to the Examples, below.

### EXAMPLE 1

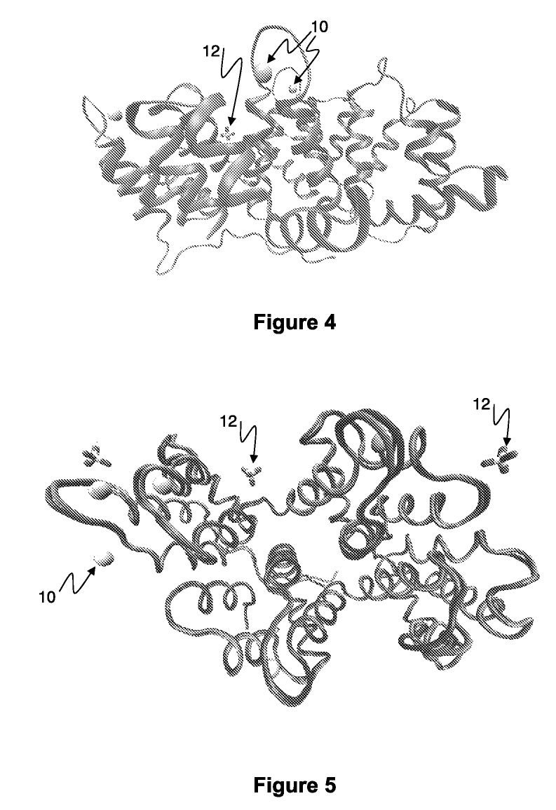

Anx-5 was crystallized in the presence and absence of divalent metal ions. X-ray diffraction quality crystals of Ca²⁺- and Zn²⁺-bound forms of avian Anx-A5 and human Anx-H5 were grown and transferred to artificial mother liquors containing glycerol as the cryoprotectant, flash-frozen in liquid N₂ and diffracted at a temperature of −160° C. The Ca²⁺ bound form of Anx-A5 crystallized in space group R3 (primitive rhombohedral) and diffracted to a resolution of 1.8 Å with unit cell dimensions of a=97.98, b=97.98, c=94.52 Å, α=90, β=90, γ=120 degrees. The structure, solved using the molecular replacement method and refinement, is shown in the helix tube plot seen in FIG. 4 from the side of the convex upper surface. Seen in FIG. 4 are spherical Ca²⁺ ions 10 as well as tetrahedral SO₄²⁻ ions 12. The convex surface of Anx-A5 has three projecting loops in domains I, II, and IV that contain the Ca²⁺ binding sites and are believed to interact with the lipid bilayer. The positions of the bound SQ₄²⁻ ions 12 represent what are believed to be potential binding sites for the phosphate moieties of phospholipids. Crystals of Anx-A5.Ca²⁺ yielded 1.4 Å resolution, the highest of any in the Protein Data Bank (PDB ID: 1YII).

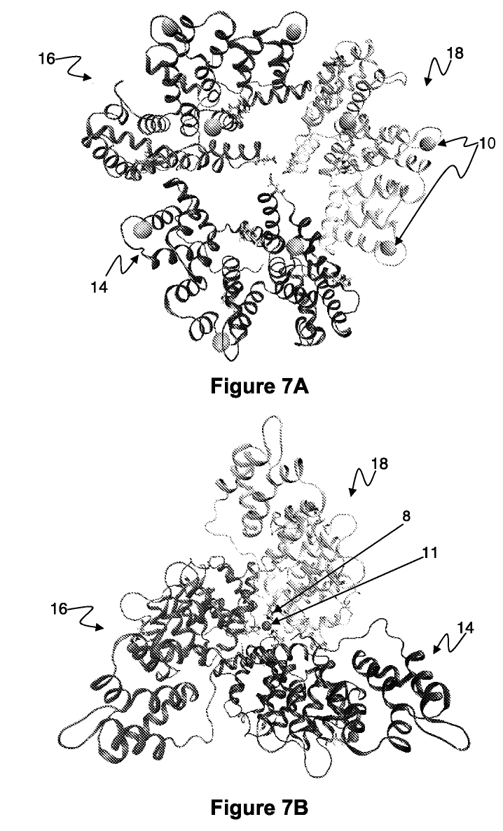

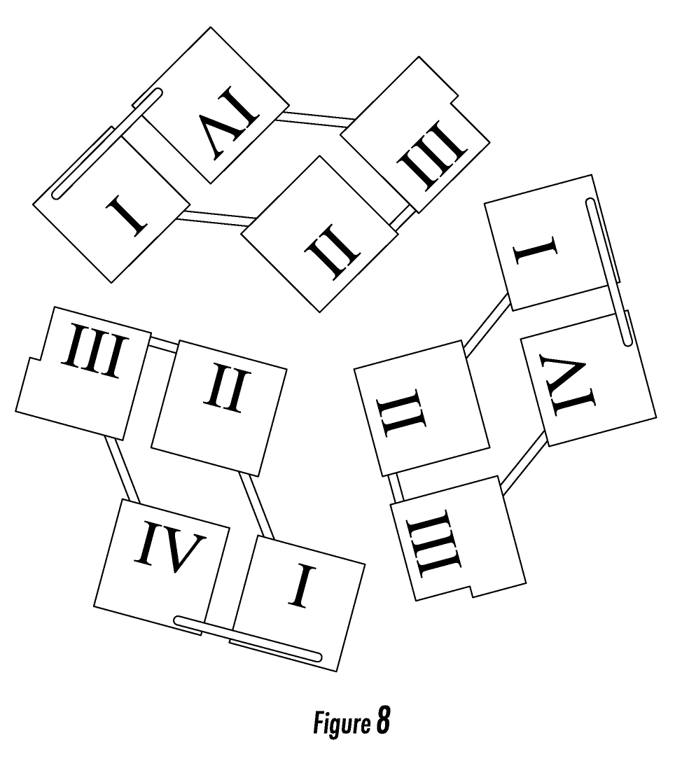

Utilizing diffraction quality crystals of native Anx-H5, crystal structures of ligand-bound forms of human Anx-H5 were compared with the more fully studied avian Anx-A5 form. As expected from sequence homology, Anx-H5 has essentially the same backbone as native Anx-A5 (FIG. 5). Each of the four repeats in the core domain contains five α-helices; helix-loop-helix folds in each of the four repeats, and the eight loops formed by these motifs are on the convex face of the disk. These helix-loop-helix folds form loops that mediate Ca²⁺-dependent binding to the periphery of membranes by a bridging mechanism in which coordination sites for Ca²⁺ are provided by the protein and by the phosphatidylserine polar head group. The face of the soluble protein that contains the Ca²⁺ sites is slightly convex, and it is thought that small inter-domain movements occur upon membrane binding to allow the Ca²⁺ sites to become coplanar. A vertical view of the avian Anx-A5.Ca²⁺ trimer form is shown in FIG. 7A and a schematic illustration of the trimer including the interrelationship of the domains is shown in FIG. 8.

### EXAMPLE 2

Zn²⁺ causes Anx-5 to bind to acidic phospholipids with even higher affinity than does Ca²⁺; however, its effect is very different. For example, low levels of Zn²⁺ (5 μM) in SCL completely inhibit MV mineralization, whereas addition of 1,10-phenanthroline (OP), a metal ion chelator, to Zn²⁺-inhibited MV restored the ability to mineralize.

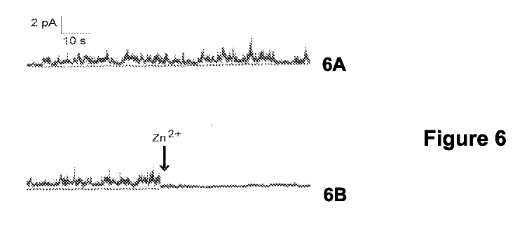

To determine if the inhibitory effect of Zn²⁺ on MV calcification was due to interaction with Anx-A5, experiments were conducted to investigate the nature of the MV Ca²⁺ channel. It was found that Ca²⁺ channels in MV have essentially identical characteristics to those of highly purified Anx-A5. When MVs or purified Anx-A5 were fused with planar PS.PE (1:1) bilayers, a multiconductance Ca²⁺ channel was evident, as shown in FIG. 6A. However, as can be seen with reference to FIG. 6B, lower graph, Ca²⁺ currents were blocked by Zn²⁺ as is Ca²⁺ uptake by MV incubated in SCL. The finding that Anx-A5 Ca²⁺ channel gating is modulated by Zn²⁺ raised the possibility that it may be an important regulator of MV Ca²⁺ channel activity in vivo. Thus, it was desirable to determine how Anx-5 helps promote MV mineralization and how Zn²⁺ affects the structure of Anx-A5.

It was found that Anx-A5 crystallized in the presence of Zn²⁺ showed a distinctly different space group (cubic) than Anx-A5 crystallized in the presence of Ca²⁺ (orthorhombic), as can be seen with reference to FIGS. 7A and 7B. The unit cell dimensions of Anx-A5 crystallized in the presence of Zn²⁺ were found to be a=106, b=106, c=106, α, β, γ=90 degrees, which were also different. These crystals of Anx-A5.Zn²⁺, analyzed at the U.S. Department of Energy's (DOE) Argonne National Laboratory facility, diffracted at 2.5 Å resolution. Determined by the molecular replacement method, the structures of the Zn²⁺ binding sites were completely different from those of the Ca²⁺ form; binding of Zn²⁺ led to dramatic reorganization of the Anx-A5 trimer as evidenced in FIG. 7B. It is further demonstrated that Zn²⁺ inhibition of the MV Ca²⁺ as seen in FIG. 6 is due to the effect of Zn²⁺, which forms a less planar, distorted globular arrangement of the avian Anx-A5 trimer, and has a different orientation with respect to the membrane than the Ca²⁺-form.

Referring to FIGS. 7A and 7B, the protein backbone of each subunit is shown 14, 16, 18 with Ca²⁺ ions 10 in FIG. 7A and Zn²⁺ ions 11 in FIG. 7B. The monomers of the physiological Anx-A5.Ca²⁺ trimer (FIG. 7A) are arranged such that the putative Ca²⁺ ion channels within each monomer are perpendicular to the three-fold axis. Three large open domains are evident at the center of the planar Anx-A5.Ca²⁺ trimer (FIG. 7A). This trimer arrangement forms upon assembly of annexin 5 on supported lipid bilayers and has been observed with Anx-5 and Anx-6, but not with Anx-2. The fact that Anx-5 and Anx-6 both activate PS.Ca.Pi mineral formation, whereas Anx-2 does not, suggests that nucleation activity is dependant on this planar trimer formation. Such a configuration would position Ca²⁺ to favorably interact with both the PS and Pi to form the PS.Ca.Pi complex. In contrast, the Anx-A5.Zn²⁺ trimer of FIG. 7B shows a strikingly different arrangement in which Zn²⁺ is at the center of the three-fold axis, coordinated by a three symmetrically equivalent histidine residues 8. The resulting arrangement is more globular, with the Ca²⁺ binding sites no longer situated on one surface. Instead they point away from each other, allowing only one to interact with the bilayer at a time. This configuration prevents formation of a planar nucleationally-active trimer assembly.

Thus, while Zn²⁺ still promotes Anx-A5 association with PS membranes, it also affects their organization in a way that inhibits both formation of nucleational competent Anx-A5.PS.Ca.Pi complexes and calcium fluxes needed for mineral growth.

### EXAMPLE 3

Mineral formation was measured by incubating MVs (40 μg protein/ml) or complex (60 μg/ml) in synthetic cartilage lymph and assayed by a microplate-based biomineralization modeling system and assay as described herein. MVs were isolated from chicken GP cartilage. The PS.Ca.Pi complex was synthesized from emulsions of PS prepared by sonication in a synthetic intracellular phosphate buffer. Where indicated, pure native placental human annexin 5 (Anx-H5) was added.

Incubation was in pH 7.5 SCL at 37° C., which follows a characteristic sigmoid pattern in which a definable lag period precedes discernible mineral formation. There then follows a period of rapid mineral formation, a transient period when the rate declines, and an extended period at a progressively slower rate.

Results are illustrated in FIGS. 9A and 9B. The data illustrate that Anx-A5 synergistically activates this PS.Ca.Pi complex by forming a quaternary Anx-A5.PS.Ca.Pi complex prepared from Mg²⁺-containing phosphate buffer matched to the ionic composition of the intracellular fluid of GP chondrocytes. To document that the effect of avian Anx-A5 could be equally matched by human Anx-H5, the kinetics of mineral formation were examined when both avian MV and the Mg²⁺-containing PS.Ca.Pi complex were seeded into SCL in the absence and presence of purified native human placental Anx H5. To ensure that the observed increases in absorbency were caused by mineral formation and not simple coagulation or coalescence of the added nucleators, control experiments were run in which Pi-free, Ca²⁺-containing SCL was used to prevent mineral formation, but still allow potential particle aggregation. In these controls there was no increase in absorbency at 340 nm over baseline for the full incubation period. For clarity, only the MV baseline is shown (FIG. 9A). As a further control, nucleational activity of Anx-H5 without PS was examined when seeded into SCL; as is evident in FIG. 9A it does not nucleate mineral.

Mg²⁺-containing PS—Ca-Pi complex was seeded into SCL, which induced mineral formation much more slowly than did isolated MVs. As can be seen with reference to FIG. 9A, once induced, its rate was less rapid, and it did not produce as much mineral as did MVs. However, seeding the quaternary Anx-H5.PS.Ca.Pi complex into SCL led to ˜2-fold quicker, as well as ˜4-fold faster and ˜2-fold greater overall mineral formation, when compared to the Mg²⁺-containing PS.Ca.Pi complex alone. In addition, the nucleation potential (an indicator of both the speed of onset and initial rate of mineral formation) was 9.6-fold higher. Thus, addition of Anx-H5 during preparation of the PS.Ca.Pi complex transformed it from being a weak nucleator to one with the ability to induce and sustain mineral formation comparable to that of native MV. Further, addition of Anx-H5 to freshly isolated MV significantly enhanced the ability to induce and propagate mineral formation. Since addition of Anx-H5 by itself to SCL failed to induce any mineral formation, this indicates that it is the interaction between MV and Anx-H5 that enhances mineralization. The similarity of the activation of mineralization of both the PS.Ca.Pi complex and isolated MVs by Anx-H5 revealed that the PS.Ca.Pi complex serves as an excellent model of MV mineralization and that Anx-H5 (human placenta) is as potent as Anx-A5 (avian cartilage) in enhancing mineralization.

Powder X-ray diffraction data on freeze-dried MV and PS.Ca.Pi complex+human Anx-H5 after incubation in SCL for 16 h were collected on a Rigaku DMax 2100X-ray diffractometer using Cu Kα radiation and are shown in FIG. 9B. Generator power was set to 40 KV and 50 mA; samples were mounted on a glass slide. Data were collected from 5-70° 2θ with steps of 0.050° with a count time of 2.0 seconds per step and a scan speed of 1.5° per minute. X-ray diffraction patterns of the mineralized MV 100 and PS.Ca.Pi complex+human Anx-H5 110 show a poorly crystal-line HAP-like mineral phase, similar but less crystalline than mature bone 120. Diffraction of the MV or PS.Ca.Pi complexes before incubation in SCL showed no evidence of the presence of crystalline mineral 130.

### EXAMPLE 4

Co-crystallography of Anx-H5 with several endogenous ligands was carried out. Large quantities of both native avian, and a recombinant form of human Anx-H5, based on the Anx-H5 pETBlue-1 (pJ123) vector from Jonathan Tait, U. of Washington, Seattle) were sent to the High Throughput Screening Laboratory at the Hauptman-Woodward Medical Research Institute located in Buffalo, N.Y. Crystallization screens of these proteins were set up in multiple 1536-well plate formats. Each well contained either native chicken Anx-A5, or the recombinant human Anx-H5 protein sample, paraffin oil and one of 1536 different combinations of a cocktail solution containing various precipitants, concentrations and buffer types over broad pH range. After two weeks, over 9000 individual images were screened for conditions conducive for generating X-ray diffraction quality Anx-5 crystals—with its natural ligands (Zn²⁺, Ca²⁺ and Pi) bound. Wells containing crystals were then matched to the specific cocktail composition. Those conditions were then duplicated in order to grow larger X-ray diffraction-quality crystals for structural analysis.

Crystal twinning proved to be a consistent problem with the recombinant version of human Anx-H5. Therefore, attention was redirected to growing crystals of native Anx-H5 isolated from human placenta; it was co-crystallized with various potential natural ligands. From the in vitro modeling studies described above and crystallography studies, the principal low MW ligands for Anx-5 were found to include Ca²⁺, Pi, and Zn²⁺. Despite the importance of PS, because of its size and amphipathic nature, it is not conducive to obtaining crystals for X-ray diffraction. Instead, O-phosphoserine, the water-soluble head group portion of PS was used to help identify the PS binding site in human Anx-H5. Protein crystals of native human Anx-H5 in many different combinations of Ca²⁺, Zn²⁺, and O-phosphoserine have been grown. The best X-ray diffraction data so far obtained at the Advanced Photon Source at the Structural Biology Center at Argonne National Laboratory are ˜2.2 Å resolution.

To determine the nature of the native PS.Ca.Pi complex, molecular modeling and computational chemistry were used to gain a more detailed understanding of how the complex is formed and nucleates mineral formation during MV-mediated mineralization. Biomimetic methods for the synthesis of mineral-inducing complexes to reconstruct the functional machinery of MV were also developed. Together, these in vitro and in silico models of mineralization provide a powerful complement to the crystallography studies.

The modeling experiments were built by exploring the physico-chemical nature of the initial mineral phase present in MV. In these studies, MV were analyzed directly by solid-state ³¹P-NMR, or incubated with hydrazine or NaOCl to remove organic constituents. The nucleational core was analyzed using TEM, EDAX, electron- and x-ray-diffraction, FTIR spectroscopy, HPTLC and SDS-PAGE. It was found that most of the MV proteins and lipids could be removed without destroying the nucleation core; however, treatment with NaOCl annihilated all activity. SDS-PAGE showed that Anx-A5 was the major protein in the nucleation core. HPTLC revealed that the detergents removed the majority of the polar lipids, but left significant free cholesterol and fatty acids, and small but critical amounts of PS. TEM showed that the nucleation core was composed of clusters of 0.7-0.8 nm diameter subunits which EDAX revealed contained Ca²⁺ and Pi with a Ca/P ratio of 1.06±0.01, indicative of the presence of acid phosphate. Electron diffraction, X-ray diffraction, and FTIR all indicated that the nucleation core was noncrystalline. ¹H-cross-polarization ³¹P-NMR indicated that the solid phase of MV was an HPO₄²⁻-rich mixture of amorphous calcium phosphate (ACP) and a complex of PS, Ca²⁺ and Pi. Using radial distribution function X-ray absorption fine structure (RDF-EXAFS) determined interatomic distances between Ca²⁺ and key atoms of the PS.Ca.Pi complex were determined.

Using molecular modeling, the epitaxial processes involved when the PS.Ca.Pi complex and ACP components of the nucleational core first form and then transform to the first crystalline structure have been visualized for the first time. Based on information presented above, computer simulations were developed to determine the molecular structure of the simple PS.Ca.Pi complex. FIG. 10 presents illustrations of computer generated molecular models of PS and its rearrangement upon Ca²⁺ and Pi binding to form the PS.Ca.Pi including ball-and-stick and licorice models (FIG. 10A) and a model of the polar head group of PS with Ca²⁺ and Pi bound to key functional groups (FIG. 10B). The stoichiometry of the synthetic complex (PS.Ca.Pi, 1:1:1) is based on direct chemical and EDAX analyses. Since the complex forms only if Pi is present in excess with PS when Ca²⁺ is introduced, it can be assumed that Pi initially interacted with the —NH₃⁺ group of the serine moiety. Further, upon addition to the PS-Pi mixture, Ca²⁺ must interact with Pi, as well as the bridge phosphoryl and the carboxylate oxygens of the PS polar head group.

To explore this interaction using quantum chemical calculations, the partial charges of the atoms of the PS head group were refined using ab initio quantum chemical calculations using Gaussian 03W (Gaussian, Inc., Wallingford, Conn. 06492) and Trident 1.0.0 (Wavefunction, Inc., Irvine, Calif. 92612) software, using O-methyl-L-serine phosphate anion as the model compound. Its geometry was first optimized in water; then using a stepping-stone approach the geometries and the partial charges of the PS head group atoms were determined using the Hartree-Fock/3-21 G and Hartree-Fock/6-31 G* basis sets and then sequentially optimized in the absence and presence of Ca²⁺ and PO₄³⁻. As shown in FIG. 10B, these calculations reveal that deprotonation of the PS—NH₃⁺ occurs when H⁺ is displaced 2.6 Å (arrow) toward the PO₄³⁻ oxygen.

### EXAMPLE 5

Molecular dynamic (MD) simulations of multiple subunit PS.Ca.Pi complex formation were made to ascertain more accurately how the complex forms, as well as to gain insight into its atomic structure. At pH 7.5 in aqueous media, the dominant form of phosphate is HPO₄²⁻ (pKa of H2PO₄²⁻=7.23). However, it is well established that binding of Ca²⁺ to HPO₄²⁻ causes it to deprotonate. Classical thermodynamic calculations and ab initio simulations reveal that Ca²⁺ causes deprotonation of HPO₄²⁻ by causing formation of a tri-ionic complex, [Ca²⁺.HHPO₄²⁻.Ca²⁺]²⁺ which readily deprotonates to form [Ca²⁺.PO₄³⁻.Ca²⁺]⁺. This tri-ionic complex can progress toward aggregation of calcium phosphate in solution. In addition, early 13C- and 31P-NMR studies have also shown that Ca²⁺ binding causes deprotonation of the —NH₃⁺ group of PS. Our quantum chemical calculations of the structure of PS.Ca.Pi complex provide insight into the mechanism of deprotonation.

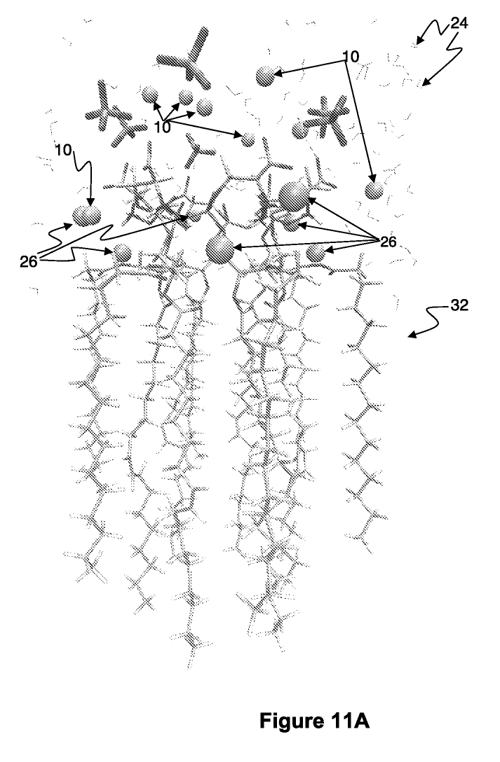

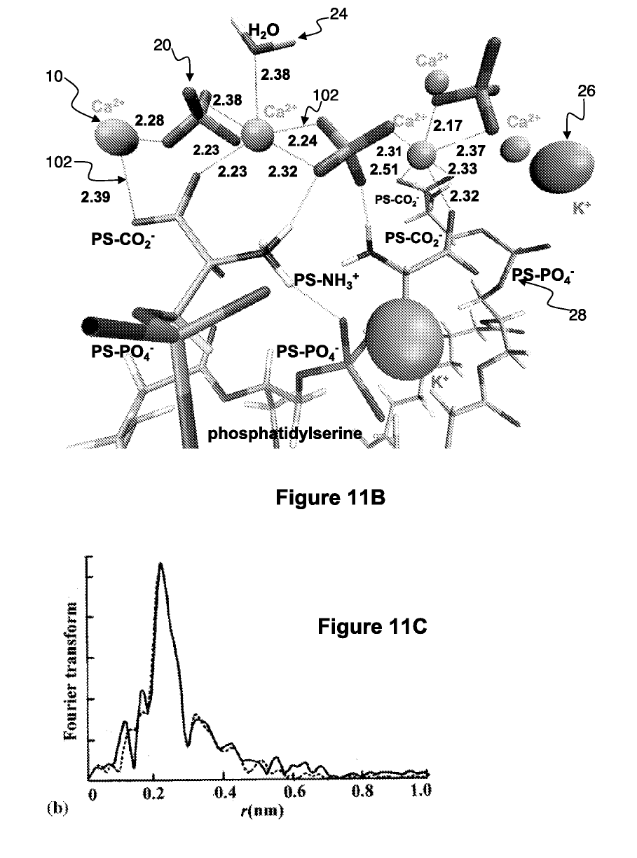

To simulate the interactions that occur between Ca²⁺ and Pi ions and several PS molecules in a membrane, the starting configuration was arbitrarily set as a loose hexagonal arrangement of 6 PS molecules in a monolayer below a random arrangement of 9 Ca²⁺, 6 PO₄³⁻, 6 K⁺ and a large number of water molecules, which roughly approximated conditions used for in vitro studies. It was constructed using VMD software. FIG. 11A illustrates this initial configuration. The polar head groups of PS are shown above the acyl side chains and are beneath the bound Ca²⁺ and Pi ions. The negatively charged carboxylate and phosphodiester moieties, and the positively charged ammonium group of the polar head of each PS molecule were allowed to interact with Pi and Ca²⁺ ions in the presence of water. After 2 ns of molecular dynamic simulation at 310 K, a snapshot taken from a lateral view of the system reveals an extensive network of Ca—O bonds 102 (FIG. 11B). For clarity, only a portion of the magnified array is shown, consisting of five of the Ca²⁺ ions (10), of which two display six-fold coordination to oxygen atoms (22) of the carboxylate group of PS 32, the free PO₄³⁻ (20) and water (24). Further stabilization of the complex results from inter- and intramolecular hydrogen bonds (104) that form between the PS 32 polar head group serine amino hydrogens and the oxygen atoms of the free PO₄³⁻ (20) as well as the oxygen atoms of the phosphodiester groups 28 of PS 32. Some of the computed Ca—O bond lengths after simulation are indicated in FIG. 11B. Where direct comparison can be made, these are in good agreement with RDF-EXAFS measurements for the PS complex and associated ACP, as shown in FIG. 11C.

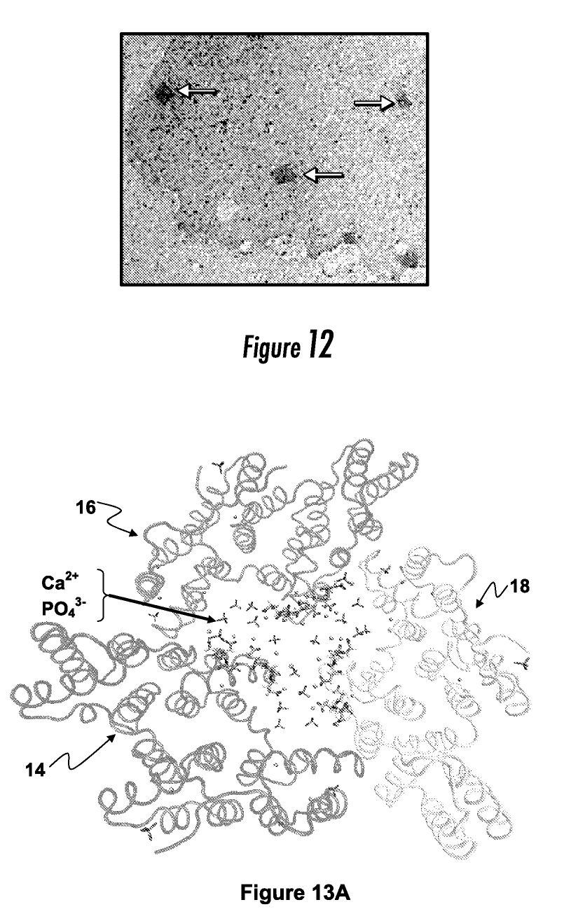

Through utilization of disclosed methods and systems it is possible to obtain accurate information on how this complex begins to establish local order, and to assemble into the quasi-crystalline array at the lipid monolayer-aqueous interface evident from the TEM studies as illustrated in FIG. 12. These methods can also be extended to larger ensembles to elucidate long-range order. Through manipulation of the in silico system, e.g., dehydration, rearrangement, and surface electrostatic attraction of solution ions, an array can be formed that is epitaxial to OCP (or HAP) crystal formation as illustrated in FIG. 1. Larger models and simulations of longer time scales provide atomistic detail of these processes.

### EXAMPLE 6

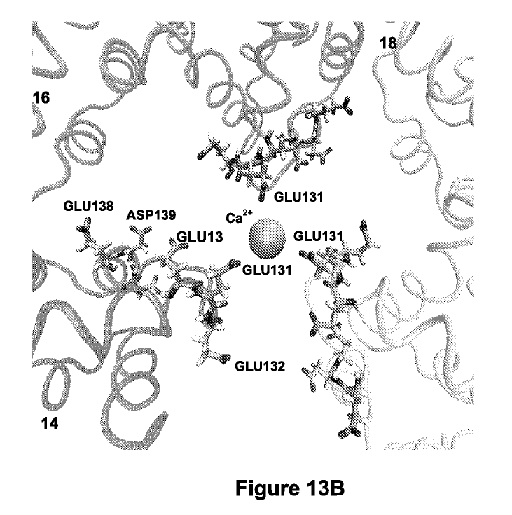

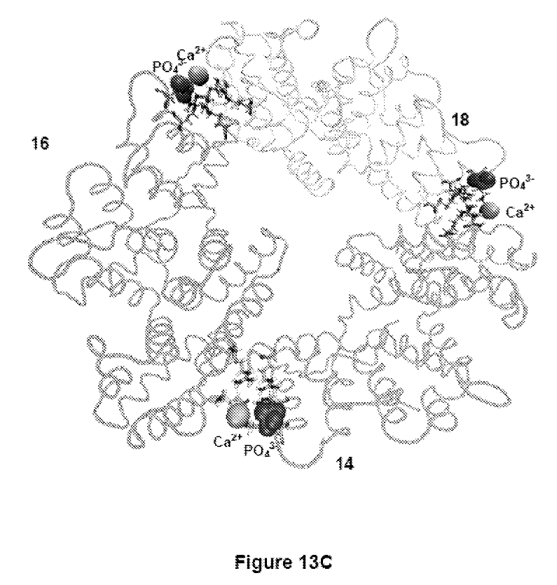

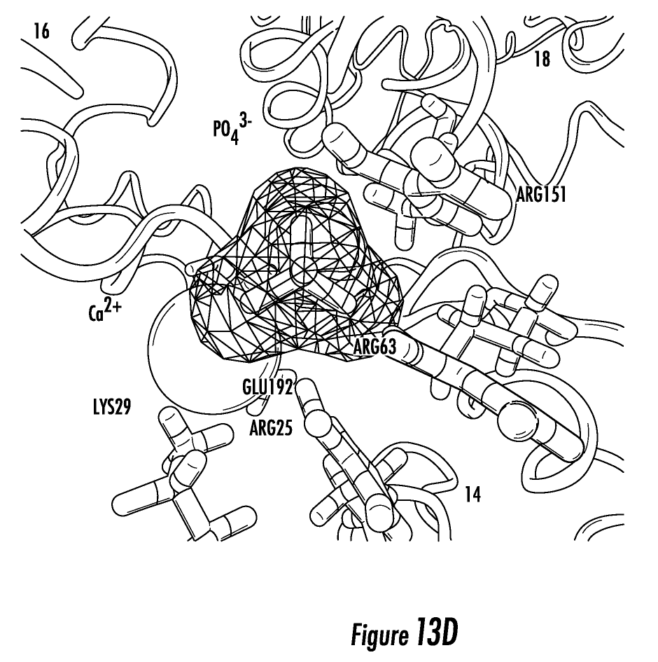

Anx-5 is monomeric in solution, but upon Ca²⁺-dependent binding to PS.PC monolayers, forms highly ordered 2 crystalline configurations. Two main types of arrays have been described, with either p6 or p3 symmetry. The p6 form occurs when the PS content of the planar membrane is low (5-20%), such as occurs in native MV; whereas the p3 form occurs when the PS level is high (>40%). Atomic force microscopy studies indicate that the p6 type forms first, but the two forms are reversible and stable. The marked stimulation in the rate and extent of mineral formation when Anx 5 is incorporated into the PS.Ca²⁺.Pi complex results from its ability to form 2-D crystalline arrays that facilitate nucleation and growth of crystalline Ca-Pi mineral. There is a high electrostatic potential of the series of negatively charged residues (GLU130-132) in Anx-H5 that lines the internal “clover leaf”-like hole created by Anx-5 trimer formation, as can be seen in FIGS. 13A-D, which include tube drawings of the inner structures (FIGS. 13A and 13B) of the Anx-H5 trimer showing the rich acidic amino acid environment in the interior space and outer contact points (FIGS. 13C and 13D) of the Anx-H5 trimer showing the array of cationic amino acid residues with Pi and Ca²⁺ binding sites where nucleation of mineral is believed to occur. From the crystal structure of Anx H5, molecular modeling as described herein has revealed that rotation of the GLU131 residues from the three monomers toward the center of the trimer hole forms a potential strong hexa-coordination binding site for a Ca²⁺ (FIG. 13B). This cation-binding site is believed to not only help promote trimer formation, but is also positioned to facilitate Pi binding. Comparing the internal volume of the basic p6 crystal form (˜15,000 Å3) with the crystal lattice parameters of OCP (a/2=9.94 Å, b=9.63 Å, γ˜109o) [171] or HAP (a=b=9.36 Å, γ=120o) suggests that up to 9 hexameric clusters of PO₄³⁻ and Ca²⁺ ions in the unit cells fit into the clover-leaf space within the Anx 5 trimers. Thus, by forming trimers, Anx-5 can impose planarity and provide a charged internal surface with anionic residues spatially arranged to bind Ca²⁺ (and subsequently Pi) ions in a manner conducive for promoting nucleation (FIG. 13A). The unique internal volume created by Anx-5 trimer formation, and the fact that Mg²⁺ does not bind to Anx-5, can also facilitate nucleation of calcium phosphate mineral at that site.

Alternatively, through disclosed modeling systems, it has been found that Ca²⁺ and PO₄³⁻ binding sites can be based on features of the outer surface created when Anx-5 forms trimers (FIGS. 13C and 13D). A general feature of the crystal structures of various species of Anx-5 trimers shows the formation of strong salt bridges between acidic and basic amino acid side chains of adjacent subunits. The mechanism of Anx-5 trimer formation and its adsorption to phospholipid membranes has been investigated previously. Several rat Anx 5 single-point mutants were created, targeting this “interfacial basic cluster”—a series of spatially proximal basic residues at sites of subunit interaction. None of the mutants significantly affected the global tertiary structure of the monomeric protein; i.e., only local changes in charge density were imparted in the immediate vicinity of these mutations. This allowed direct observation of the effect of mutation on trimer formation. Specifically, the rat ARG23→GLU23 mutation was found to be most effective at inhibiting adsorption of Anx-5 to the membrane, suggesting that this region is critical for binding to phospholipids. Of particular interest from the perspective of searching for possible sites of calcium phosphate nucleation, are potential phosphate binding sites suggested by bound sulfate ions in Anx 5 crystal structures. Structures of wild-type Anx-5 crystallized from sulfate-containing solutions have previously revealed a sulfate ion near the primary coordination shell of Ca²⁺ in the IIIAB loop. Further, rat Anx-5 crystallized in the presence of both Ca²⁺ and head-group analogs of PS formed a ternary complex in which the phosphate ester group occupied the sulfate site. Therefore, the region near this site in human Anx-H5 (FIGS. 13A and 13B) is an additional locus in the quaternary complex where Ca²⁺ and PO₄³⁻ can bind in a spatial arrangement conducive for nucleation. Thus, drugs targeted to any of these regions would inhibit trimer formation and thus prevent the onset of mineralization in pathological calcifications or other membrane mediated disease process such as inflammation and cancer that involve annexin 5. Furthermore, any small molecule drug, antisense DNA, antisense RNA and RNAi that can diminish expression of Anx-H5, or enzymes involved in the synthesis of PS could be used as therapeutic agents that could reduce pathological mineralization in the body.

Presently disclosed molecular modeling systems have demonstrated, at the molecular level, the Ca²⁺-dependent binding of human Anx-H5 with PS. Site-directed mutagenesis, computer analyses, and other lines of evidence indicate that domain 1 of Anx 5 is essential and sufficient to initiate the Ca²⁺-dependent binding of the protein to PS-containing membranes. The enzyme, protein kinase Cα, shows Ca²⁺-dependent binding to membranes that is also highly specific to PS. For both it and Anx-H5 alone without PS, intrinsic binding affinity for Ca²⁺ is low; for both, PS greatly potentates the overall affinity for Ca²⁺. In addition, there are solved crystal structures of PS-like moieties bound to protein kinase Cα. Further, evidence from the crystal structural of rat Anx-A5.Ca²⁺ complexes with PS head group moieties indicates that a novel “Ca²⁺-bridging” mechanism exists for binding to interfacial membrane proteins such as protein kinase Cα and the annexins. Unique binding sites for phospholipid head groups, which have been proposed to be PS receptor sites in vivo have also been observed.

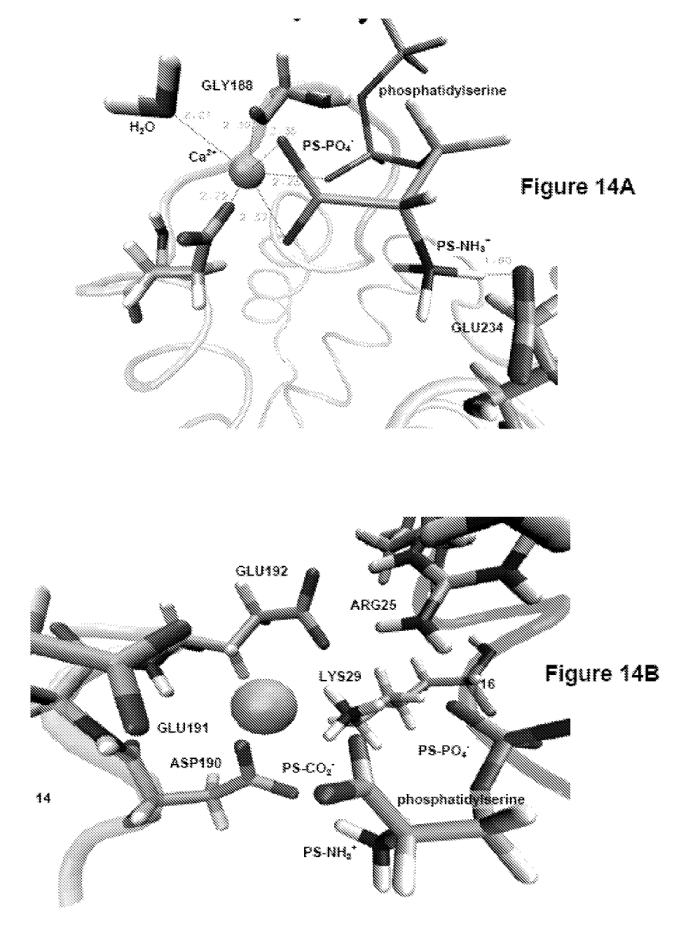

Based in part on the in silico discovery of a new PS-binding site in rat Anx 5 domain 1 (Arg25-X3-Lys29 . . . . Arg63-X4-Asp68-X2-Ser71-Glu72) by others (see Montaville et al. J. Biol. Chem. 277:24684-93, 2002), a first approximation model of the structure of the two putative human Anx-H5.PS binding sites has been developed according to disclosed modeling methods, as illustrated in FIG. 14. From crystallographic data obtained, the disclosed model differs from that of previous researchers in two respects: 1) X-ray crystallography shows that the Ca²⁺ is coordinated by a sulfate/phosphate group in the axial position. 2) Only one molecule of PS, rather than two, is included in each binding site. Based on the approach described previously, the phosphoryl moiety of the polar head of the first PS was positioned in the purported binding region between helices A and D of domain 1 in Anx-H5, very near to where a sulfate group is observed in the crystallographic structure (see FIG. 4). The phosphoryl group of the second PS was positioned near the other sulfate and proximal to the Ca²⁺ ion.

Using the disclosed modeling systems and methods, including developed parameters calculated for PS via quantum mechanics as described herein, a 100 ps molecular dynamic simulation shows synergistic binding between Ca²⁺ and GLU191, GLY188 and the carboxyl and phosphoryl oxygens of PS, with GLU234 forming a H-bond with the ammonium hydrogen of PS (FIG. 14A). The second PS binding site was coupled to Ca²⁺ bounded by GLU181 and GLU192 and by the carboxyl and phosphoryl oxygens of PS that are also coordinated with the amino hydrogen of LYS29; ASP190 forms a H-bond with the ammonium hydrogen of PS (FIG. 14B). Disclosed models reveal atomic scale precision details of Anx-H5.phospholipid bilayer interactions, which greatly increases understanding into the association of membrane proteins with the lipid bilayer. Used in conjunction with the previously described crystallography, disclosed in silico and in vitro mineralization models shows structure and function correlates that have elucidated and teach the critical first steps by which Anx-H5 and PS.Ca.Pi complex nucleate calcium phosphate mineral formation.

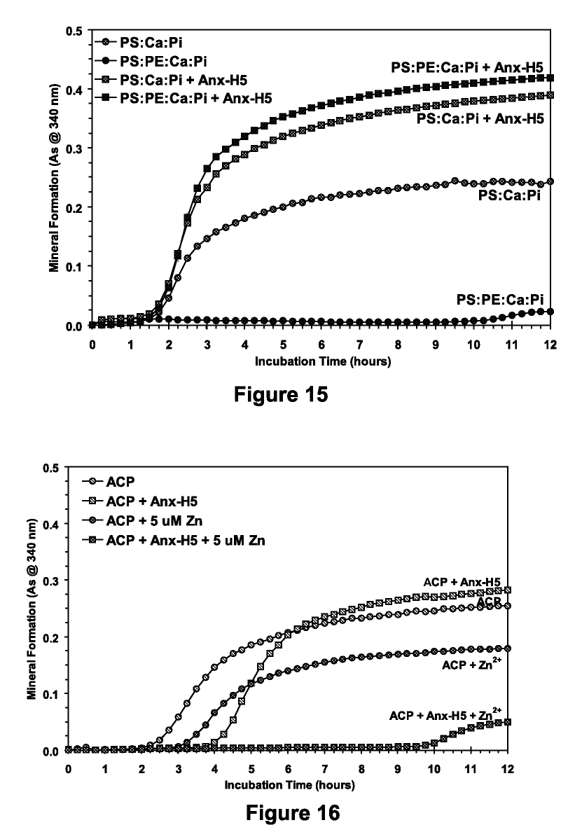

FIG. 15 illustrates the effect of Anx-H5 on mineral formation by synthetic PS and PE containing Ca.Pi complexes as determined through in vitro modeling of the system. Mineralization was measured by incubating the various complexes as shown (about 50 μg/ml) in SCL using a microplate-based biomineralization assay system. The complexes were prepared as described above in Example 3. Emulsions of PS or PS.PE (1:1) were generated by sonication to generate approximately 60 nm vesicles. Anx-H5 was added before CaCl₂ was titrated to form the complex.

As can be seen with reference to FIG. 15, while incorporation of Anx-H5 into the PS.Ca.Pi complex caused an obvious stimulatory effect, incorporation of equimolar amounts of PE into PS.Ca.Pi caused profound inhibition of its ability to nucleate mineral formation. This profound inhibitory effect is surprising because PE is an abundant lipid in the inner MV membrane. The remarkable ability of Anx-H5, the dominant protein in MV, to overcome this inhibitory effect of PE is significant. It was seen with native avian Anx A5 and is shown in FIG. 15 with human Anx H5. This ability of native Anx-5's to stimulate PS.PE.Ca.Pi-induced mineralization is believed to be an important feature of the ability of native MVs to nucleate mineral formation. Incorporation of Anx-5 enables PS.PE.Ca.Pi complexes to closely mimic the nucleational activity of native MV and supports its important role in de novo mineral formation.

### EXAMPLE 7

While the crystallographic studies show that chicken Anx-A5 and human Anx-H5 have highly homologous monomeric structures (FIG. 5), there are key differences in the amino acid sequences in the critical central region of the trimer where Zn²⁺ binding to HIS286 causes marked distortion of the normal planar structure of avian Anx-A5. Although this feature is not present in human Anx-H5, in vitro testing shows marked inhibition of mineral formation by Anx-H5-containing ACP when low levels (5 μM) of Zn²⁺ are included in SCL, as shown in FIG. 16. The strong inhibitory effect of Zn²⁺ on the induction of mineral formation by Anx-H5-containing ACP is significant. In contrast, Zn²⁺ had much less of an effect on simple ACP-seeded mineralization. Taken together, these findings indicate that Zn²⁺ is acting on the protein more than on the nascent mineral. While at high levels, Zn²⁺ is known to inhibit HA formation, presently disclosed methods indicate that low levels of Zn²⁺ cause only weak inhibition of ACP-induced mineral formation.

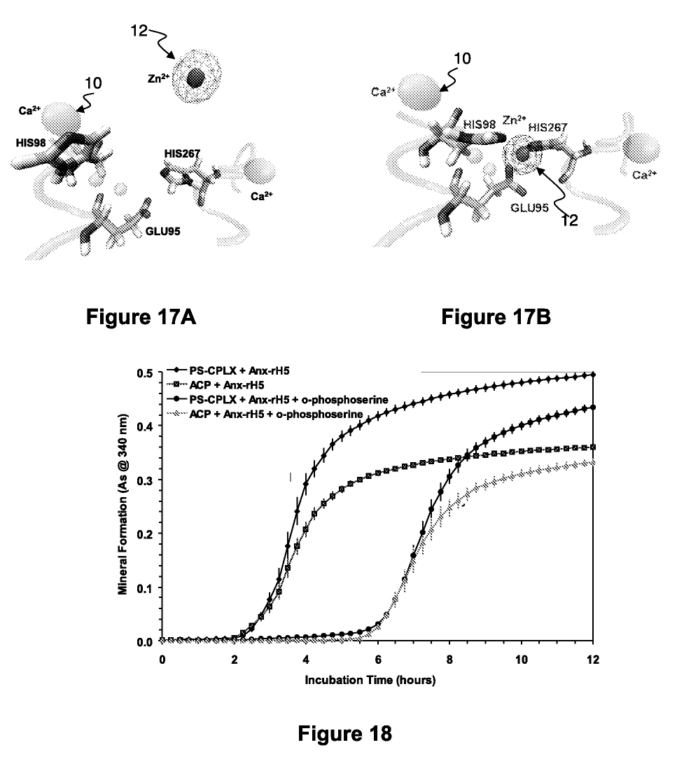

MD simulations as described herein were utilized to explore Zn²⁺-binding sites in human Anx-H5. As shown in FIGS. 17A and 17B, utilizing a computer modeling system as described above, when a Zn²⁺ ion is placed approximately 10 Å from His98 and His267 and 13 Å above Glu95 of a monomer of Anx-H5—whose proximity to each other was evident in the crystalline structure—after minimization and equilibration (FIG. 17A), time-course snapshots reveal that Zn²⁺ rapidly moves to and becomes coordinated by these residues, inducing a significant rotation in His98 (FIG. 17B). In the final metal-bound Anx-H5 structure, the histidine residues interact with Zn²⁺ via their central ring-N, as does a glutamate side-chain carboxylate-O. The geometry and bond distances revealed from these MD simulations are in good agreement with zinc coordination values.

The power of the MD simulations described herein reveals a potential Zn²⁺-binding site in human Anx-H5 that was not obvious and could not have been predicted from sequence alignment of known Zn²⁺-binding proteins, or from the crystal structures. The inhibitory effects of Zn²⁺ in markedly slowing induction of mineral formation show that it is useful in preventing unwanted de novo mineralization. Combining the Anx-H5.Zn²⁺ crystallography with the in vitro testing systems described herein reveals that Zn²⁺ can be incorporated into lavage rinses used during joint arthroscopic surgeries. Therefore, it could be used as a cost-effective measure for inhibiting articular cartilage mineralization.

### EXAMPLE 8

Phosphoserine was examined as a test compound. Phosphoserine can occupy PS head-group binding sites in Anx-H5, acting as a competitive inhibitor of PS binding. Unlike PS, phosphoserine does not contain acyl chains, making it unable to form bilayers. Since Anx-H5 trimer formation occurs only on phospholipid bilayers, phosphoserine competes for PS binding sites in Anx-H5, interfering with trimer formation, and thereby inhibiting de novo mineralization. To demonstrate this, Anx-H5.PS.Ca.Pi complexes were synthesized and the kinetics of mineralization was measured in the absence or presence of 100 μM phosphoserine. Results are shown in FIG. 18. Evident is the fact that phosphoserine significantly delayed induction, almost tripling the time needed for onset of mineralization—using both Anx-H5-containing ACP and PS.Ca.Pi complexes. The finding that O-phosphoserine delays the onset of mineralization shows that it may also compete for Pi-binding sites in Anx H5, potentially at the spatially proximal series of basic residues (Arg25, Lys29, Arg63, Arg151) at the subunit interface of the Anx-H5 trimer (FIG. 14B).

FIG. 18 illustrates the results of in vitro assays showing the delay of onset of mineralization of Anx-H5 containing nucleators by incorporation of O-phospho-L-serine

### EXAMPLE 9

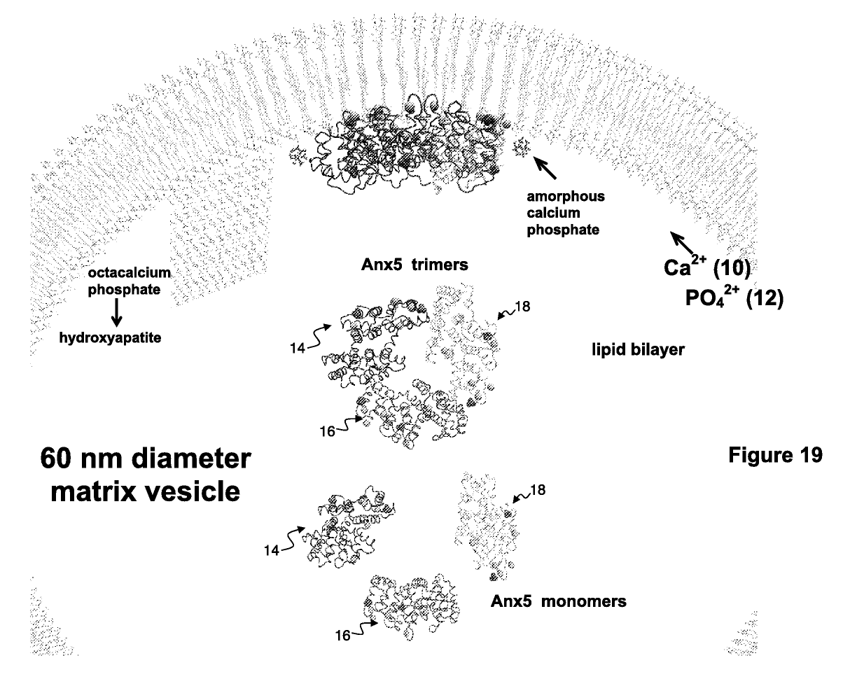

Having identified the essential components of the MV nucleational core required for nucleation both biochemically and by parameterized computation, larger scale biological systems of mineralization were assembled. Exploration of the kinetics of these complex interactions is not feasible using standard biochemical and crystallographic methods. Therefore, molecular dynamic (MD) simulation as described herein were used to demonstrate the atomistic mechanism by which human Anx-H5 interacts with phospholipid bilayers in the presence of Ca²⁺ and HPO₄²⁻. As illustrated in FIG. 19, a nanoscale segment of the MV nucleational core including PS 32, Ca²⁺ 10, HPO₄²⁻ 34, K⁺ 26 and H₂O 24, was constructed and visualized using Tool Command Language scripts run with Visual Molecular Dynamics (VMD) software. The segment of a 60 nm diameter bilayer assembly (similar in size to MV) containing hundreds of PS molecules 32 was first created with the phosphodiester phosphate atoms of the head group approximately 8 Å apart in a loose arrangement and is illustrated in FIG. 19. To mimic the basic unit of ACP, several clusters of 9 Ca²⁺ 10 and 6 PO₄³⁻ 20 atoms were added in a random arrangement 5-7 Å below the PS 32 head groups. A trimer of human Anx-H5 14, 16, 18 was positioned 12-15 Å below the bilayer. Next, since these complexes are formed in vivo in K⁺-rich solutions, to neutralize the negative charge of the system, K⁺ atoms 26 were added to the ensemble. Finally, the system was hydrated by adding bulk H₂O 24 above the hydrophobic region of PS 32. The complete system contains 70,000 atoms. To perform molecular dynamic simulations on a system this large, even a powerful quad core system is inadequate considering the required computational time. Therefore a TeraGrid was used (Award No. MCB070071T). This award allocation allows TeraGrid-wide-roaming access to supercomputing facilities at sites such as the Pittsburgh Supercomputing Center, which includes specific access to Bigben, a Cray XT3 machine with 4136 processors. High-performance molecular dynamics simulations were run using these TeraGrid resources provided by NSF and administered by the Pittsburgh Supercomputing Center. The system was minimized to remove unfavorable Van der Waals contacts using the conjugate gradient algorithm of NAMD for 5000 steps, then warmed and equilibrated at 310 K using a 2.0 fs time step. After the 5 ns simulation, calcium and phosphate-oxygen bond distances were in excellent agreement with Ca—O distances of RD-EXAFS data obtained for mineralizing PS-complexes and with that of the mineral phase of bone.

Thus, in silico models as described herein can be used as reliable and powerful methods to predict future mineralization events and to screen for drugs that have the potential to inhibit self assembly of the quaternary complex of PS, Ca²⁺, Pi and Anx-H5.

### EXAMPLE 10

The effect of antisense RNA to interfere with the mineralization activity of annexin-5 in an avian chondrocyte culture system was demonstrated.

A rapidly mineralizing growth plate culture system using a programmed sequence with a combination of growth factors and hormones for modeling the processes occurring during longitudinal bone growth and fracture repair and for development of tissue engineered cartilage for replacement of diseased cartilage was developed. It was found that by carefully programming the timing and duration of drug addition, profound stimulation of chondrocyte mineralization and proteolgycan expression can be induced. This culture system mimics precisely sequence of developmental events known to take place in human growth plate cartilage in vivo.

To move the discovery process toward clinical application, a retrovirus expressing antisense RNA to annexin 5 that reduces levels of functional annexin 5 was tested directly in cell-mediated mineralization systems. These studies show that these agents that can reduce levels of annexin 5 function or expression can reduce the amount of mineral formed by living cells. The strength of the described avian growth plate chondrocyte primary culture system is that it recapitulates essentially all in vivo growth plate processes, including formation of MV, and mineral deposition. It expresses phenotypic markers characteristic of endochondral mineralization: type II and X collagens, sulfated proteoglycans, alkaline phosphatase, osteonectin, and osteopontin. Further, its response to various hormonal and growth factors closely models known effects in vivo. Direct comparisons have shown very close similarities between avian, mammalian, and human GP chondrocytes. Thus, this primary culture system provides an excellent means to study factors that enhance or inhibit mineral formation by modulating annexin-5 expression.

Annexin-5 functioning as a Ca²⁺ channel to elevate Ca²⁺ influx prior to inducing GP mineralization was demonstrated. The strategy was to prepare retroviral gene delivery vehicles that produce the over-expression of normal and dysfunctional proteins in cultured GP chondrocytes. Data is presented here that demonstrates how to make the transgene constructs required for the enhanced- and loss-of-function annexin-5 protein.

Two main problems associated with the use of eukaryotic expression vectors are that stable transfection is not possible and the frequency is too low. Retroviral-mediated gene delivery has several advantages over the transient transfection achieved using conventional expression vectors. This is because gene expression is stable and the percentage of infected cells increases over time—if the virus is replication-competent. For this reason, replication-competent retroviral (RCAS) vectors based on avian Rous sarcoma virus are increasingly used to express genes in both primary cell cultures and embryonic tissues of chickens. RCAS (a gift from Constance Cepko, Harvard Medical School, Department of Genetics) was used to design and construct an RCAS-Annexin-5 expression vector.

Using the RCAS retroviral mediated gene delivery vehicle, two Annexin-5 constructs were generated, one that over-expressed the wild-type Annexin-5 gene (the “enhanced-function” construct) and one that over-expressed a dysfunctional annexin gene (the “loss-of-function” or dominant-negative construct). For the enhanced-function construct, an RCAS-Annexin-5 was prepared that contains the full-length Annexin-5 gene. For the loss-of-function construct, an Annexin-5 mutant lacking normal Ca²⁺ channel activity was prepared. The mutation was targeted to the Ca²⁺ channel pore domain where a key Glu residue was converted to Gly. The resulting loss-of-function Annexin-5 Glu→Gly mutant also has been prepared in an RCAS vector.

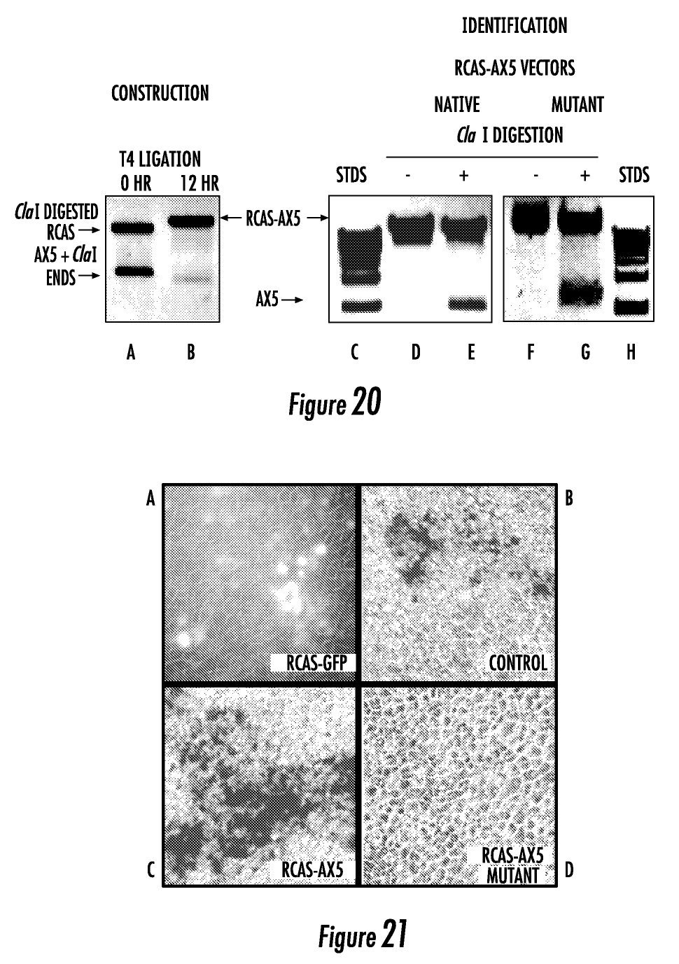

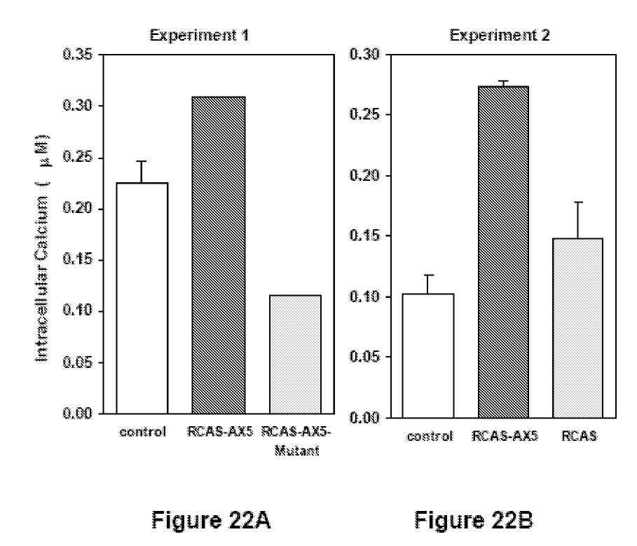

Annexin-5 was removed from a SLAX-13-Annexin-5 shuttle vector by ClaI digestion then separated from the parent plasmid by agarose gel electrophoresis. The purified Annexin-5 band was combined with the ClaI digested, alkaline phosphatase treated, heat inactivated RCAS vector (FIG. 20, lane A). The Annexin-5 band was ligated into the ClaI site of RCAS, and 2.5 μl of the resultant RCAS-Annexin-5 construct (FIG. 20, lane B) was transformed into E. coli. Plasmids isolated from colony overnight cultures were digested with ClaI to test for the presence of the ˜1 kb Annexin-5 insert (FIG. 20, lanes E and G). Positive clones were then sequenced to verify their authenticity; two of eight clones had Annexin-5 in the proper orientation in RCAS. Similarly, ClaI digestion, ligation and transformation were performed to create the RCAS-Annexin-5 (Glu→Gly) mutant, and DNA sequence analysis confirmed that again, two of eight clones containing inserts were in the proper orientation in RCAS. (On the Figure, AX5=Annexin-5.) Also prepared and used was the RCAS-native Annexin-5 and its Glu¹¹²→Gly mutant construct to infect and over-express these proteins in cultured GP chondrocytes. On day 10, cells were transfected for 7 days using GenePorter, then cultivation in fresh medium for 7 days to allow viral spreading. Results are shown in FIGS. 21 and 22.

The high efficiency of the viral promoter in RCAS-based vectors achieves sufficiently high levels of expression of Annexin-5 in the chondrocytes to overshadow normal levels of gene expression. Therefore, Annexin-5 is utilized for the influx of Ca²⁺. Thus, over-expression of native, wild type Annexin-5 by RCAS significantly increases levels of [Ca²⁺] and enhances the rate of chondrocyte mineralization. FIGS. 21 and 22 demonstrate this; they also show that overexpression of the Glu¹¹²→Gly mutant form of Annexin-5 in the GP cells leads to reduced [Ca²⁺] and no evident mineralization.

RCAS-GFP treatment produced numerous transfections shown as brightly labeled cells in dark-field (FIG. 21). In the control culture (FIG. 21) mineral deposits are evident; but more extensive mineral deposits are obvious in RCAS-Annexin-5 transfected cultures (FIG. 21). The RCAS-Annexin-5 mutant transfected culture (FIG. 21) has no evident mineral. Bar graphs illustrated in FIG. 19 show intracellular [Ca²⁺] measurements on suspensions of cells released from 2 separate transfections. In Expt. 1 (FIG. 20A), cells transfected with native Annexin-5 (RCAS-AX5) have highest [Ca²⁺], while Annexin-5 mutant transfected cells (RCAS-AX5 mutant) had the lowest. In Expt. 2 (FIG. 22), cells treated with RCAS-AX5 again had much higher [Ca²⁺] than untreated control or “empty” RCAS vector-treated cells.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole and in part. Furthermore, those of ordinary skill in the art will appreciate that the foregoing description is by way of example only, and is not intended to limit the disclosure so further described in such appended claims.

What is claimed is:

1. A method for identifying a test compound that effects formation of a complex including calcium ion, inorganic phosphate, and phosphatidylserine comprising:
integrating physical parameters of components involved in neocalcification into an in silico system, said components comprising calcium ion, inorganic phosphate, phosphatidylserine, and a test compound, said physical parameters comprising the partial charges of atoms of the components, the bond length between atoms of the components, the bond angles, and dihedrals of the components;
bringing the components within an interactive distance of one another during an in silico simulation, the interactive distance being between about 5 and about 15 Angstroms; and
determining whether the test compound blocks formation of a complex comprising the calcium ion, the inorganic phosphate, and the phosphatidyiserine.

2. The method according to claim 1, the components further comprising water.

3. The method according to claim 1, the components further comprising potassium ion.

4. The method according to claim 1, the components further comprising annexin-5.

5. The method according to claim 1, further comprising contacting the components with one another in an in vitro process.

6. The method according to claim 5, wherein the component include calcium ion, inorganic phosphate, and phosphatidylserine that are provided in the in vitro process in a matrix vesicle.

7. A method for identifying a test compound that effects neocalcification comprising:
integrating physical parameters of annexin 5 and a test compound into an in silico system, said physical parameters comprising the partial charges of atoms, the bond length between atoms, the bond angles and the dihedrals of the annexin-5 and the test compound;
bringing the annexin-5 and the test compound within an interactive distance of one another during an in silico simulation, wherein the interactive distance is between about 5 and about 15 Angstroms; and
determining whether the test compound alters the initiation of calcification by the annexin-5.

8. The method according to claim 7, wherein the test compound alters the conformation of the annexin-5.

9. The method according to claim 7, the method further comprising integrating the physical parameters of one or more phospholipids into the in silico system.

10. The method according to claim 9, wherein at least one of the one or more phospholipids is phosphatidylserine.

11. The method according to claim 9, wherein the test compound alters binding between the phospholipid and the annexin-5.

12. The method according to claim 9, the method further comprising integrating the physical parameters of multiple annexin-5 molecules into the in silico system.

13. The method according to claim 12, wherein the one or more phospholipids are components of a lipid bilayer.

14. The method according to claim 13, the method further comprising bringing at least three annexin-5 proteins and the test compound within an interactive distance of one another and the lipid bilayer.

15. The method according to claim 7, further comprising integrating physical parameters of water into the in silico system.

16. The method according to claim 7, further comprising integrating physical parameters of potassium ion into the in silico system.

17. The method according to claim 7, further comprising integrating physical parameters of one or more of calcium ion and inorganic phosphate into the in silico system.

18. The method according to claim 7, further comprising contacting annexin-5 and the test compound with one another in an in vitro process.

19. A system for determining the effect of a test compound on calcification initiators comprising:
an in silico testing protocol, the in silico testing protocol comprising physical parameters of a test compound and components involved in calcification, the components including one or more of a phospholipid, calcium ion, inorganic phosphate, annexin-5, the parameters including the partial charges of atoms, the bond length between atoms, the bond angles and the dihedrals of the compounds; and
an in vitro testing protocol, the in vitro testing protocol incorporating the same test compound and the same components involved in calcification as the in silico testing protocol.

20. The system according to claim 19, wherein the annexin-5 is mutant annexin-5.

21. The system according to claim 19, the components further comprising water.

22. The system according to claim 19, the in silico and in vitro testing protocols further comprising an additional divalent cation.

23. The system according to claim 22, wherein the divalent cation is magnesium.

24. The system according to claim 19, the components further comprising sodium or carbonate ion.

25. The system according to claim 19, wherein the test compound comprises zinc.

26. The system according to claim 19, wherein the test compound is a low molecular weight phosphate analogue.

27. The system according to claim 19, wherein the test compound is a lipid analogue.

28. The system according to claim 19, wherein the in vitro testing protocol comprises culturing the components with chondrocytes.

29. The system according to claim 19, wherein the in vitro testing protocol further comprising culturing the components in the presence of matrix vesicles.

30. A formulation for inhibiting calcification, the formulation comprising a low molecular analog of a phospholipid.

31. The formulation of claim 30, wherein the phospholipid is phosphatidylserine.

32. The formulation of claim 31, wherein the low molecular analog of phosphatidylserine is o-phospho-L-serine.

33. The formulation of claim 31, wherein the low molecular analog of phosphatidylserine is 2-amino-4-phosphonobutyric acid.

34. The formulation of claim 31, wherein the low molecular analog of phosphatidylserine has the structure: 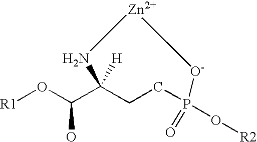
wherein
R1 and R2 are independently H, CH₃, or a substituted or unsubstituted acyl chain up to about 20 carbons in length.

35. A formulation for inhibiting calcification, the formulation comprising a low molecular analog of a phosphate.

36. The formulation of claim 35, wherein the low molecular analog is vanadate or arsenate.

37. A formulation for effecting calcification, the formulation comprising a compound including a divalent metal cation.

38. The formulation of claim 37, wherein the compound includes zinc or magnesium.

39. The formulation of claim 37, wherein the compound includes nickel, cobalt, or copper.
